(19) United States
(12) Patent Application Publication
(10) Pub. No.: US 2025/0359772 A1
(43) Pub. Date: Nov. 27, 2025

(54) Cardiac Function Assessment System

(71) Applicants: Mark Ries Robinson, Albuquerque, NM (US); Elena A Allen, Albuquerque, NM (US)

(72) Inventors: Mark Ries Robinson, Albuquerque, NM (US); Elena A Allen, Albuquerque, NM (US)

(73) Assignee: Medici Technologies, LLC, Albuquerque, NM (US)

(21) Appl. No.: 19/238,022

(22) Filed: Jun. 13, 2025

Related U.S. Application Data
(63) Continuation of application No. PCT/US22/82192, filed on Dec. 21, 2022.

(51) Int. Cl. A61B 5/026; A61B 5/00; A61B 5/024; G16H 10/60; G16H 50/30

(52) U.S. Cl. CPC A61B 5/0261; A61B 5/02416; A61B 5/7275; G16H 50/30; G16H 10/60

(57) ABSTRACT

An assessment of basal cardiac fitness uses noninvasively obtained physiological measures to determine the presence of a basal physiological state and cardiometric measures to determine cardiac fitness. In the presence of preload independence and cardiac vagal control, cardiometric signals are acquired that are sensitive to cardiac function and include information indicative of the opening and closing of the individual's aortic valve. The cardiometric signals are analyzed by a cardiac fitness assessment system for the determination of basal cardiac fitness. The invention assures preload independence through the assessment of observational physiological parameters or the use of physiological parameters obtained following alterations in venous return. The invention assures the presence of cardiac vagal control by assessment of measured parameters, including the presence of respiratory sinus arrhythmia. The resulting test is specific for cardiac function without dependencies on physical activities associated with heart rate elevation or respiratory capabilities.

### TECHNICAL FIELD

The present invention relates to determining an individual's basal cardiac fitness based on physiological and cardiometric signals obtained during a single measurement period or based on multiple measurements over time. The noninvasive system enables testing at home or in the clinic. The assessment method does not require an elevation in heart rate and is specific for cardiac function without respiratory dependencies.

### BACKGROUND

The determination of an individual's basal cardiac fitness in a convenient manner has multiple applications for elderly patients, those in cardiac rehabilitation, and athletes. Various references are mentioned herein that facilitate understanding of the invention; each of those references is incorporated herein by reference.

Current Screening and Diagnostic Approaches. A common approach to accessing cardiac fitness is to use some form of exercise testing. An example is the 12-minute run test or “Cooper test.” Dr. Ken Cooper developed the testing in the 1960s as a way for the military to measure fitness and provide an estimate of VO2 max. (Cooper KH. A Means of Assessing Maximal Oxygen Uptake. Journal of the American Medical Association, 1968. 203:201-204.4). The run test is still used today and is a simple way to assess fitness. However, the test is not specific for cardiac fitness but rather total aerobic and musculoskeletal fitness. Additionally, the result will be dependent on the subject's ability to run, has limited applicability for the elderly, and is unlikely to be viewed as convenient.

Similarly, most evaluations of cardiac fitness involve some sort of exercise or the response to some other stimulation resulting in a stress response of the heart. The ability of the heart to respond appropriately is the basis for the determination of cardiac fitness. Multiple methodologies for assessing cardiac fitness exist and include VO2 max, PACER test, treadmill test, 3-minute step test, and medication-induced stress tests. The criteria for evaluation vary but include heart rate response, EKG assessment, power output, recovery times, and metrics associated with cardiorespiratory response. Notably, stress-type tests are both an evaluation of the patient's respiratory capability as well as cardiac capability. The ability to respond to an exercise or workload is dependent upon both lung function and cardiac function. Thus, existing tests necessitate an increase in heart rate and have an inherent dependency on respiratory function.

In addition to physical performance tests, cardiac capabilities can be inferred from measurements that evaluate heart structure and function. Echocardiography is an example of a measurement that can assess both the structural elements of the heart as well as components of ejection fraction, valvular function, etc. These tests require specialized equipment and trained personnel.

Difficulties in Cardiac Fitness Testing. Many cardiac assessment tests are based on a required physical activity such as walking, running, cycling, etc., and a significant degree of patient participation. Such tests are subject to errors in measurement due to dependencies on the patient's familiarity with the activity and ancillary physical capabilities. For example, a walking cardiac test could be adversely influenced if the patient suffers from hip pain.

Additionally, a cardiac fitness test cannot be based on a volitional “maximal effort” by the patient. Cardiac muscle, unlike skeletal muscle, cannot modulate its force generation through changes in motor nerve activity and motor unit recruitment. Therefore, there is no ability for a patient to volitionally generate a “maximum effort cardiac contraction.” In contrast, lung capabilities can be assessed by a maximal exhalation effort. Thus, the development of a cardiac-specific test must account for these nuances in cardiac physiology and limited patient control over heart function.

Use Cases for Simple Cardiac Assessment. A simple assessment of cardiac fitness has value in accessing improvements in cardiac fitness as well as deterioration in cardiac fitness.

More than 1.1 million people experience an ischemic heart event, also known as a heart attack, each year in the US. Ischemic heart disease is the leading cause of mortality in the United States. (Benjamin, Emelia J., et al. “Heart disease and stroke statistics—2017 update: a report from the American Heart Association.” Circulation 135.10 (2017): e146-e603). Cardiac rehabilitation is an evidence-based therapy that reduces mortality, morbidity, and hospital readmissions in patients with ischemic heart disease. (Aragam, Krishna G., et al. “Gaps in referral to cardiac rehabilitation of patients undergoing percutaneous coronary intervention in the United States.” Journal of the American College of Cardiology 65.19 (2015): 2079-2088). The ability to access improvements in cardiac function would be of significant value to the providers working with these patients by providing an assessment of improvement.

The assessment of cardiac fitness has value for the more than 19 million participants in endurance events, defined as exercise events lasting more than 3 hours. These individuals have a significant interest in their cardiac performance and would value a simple home test. Such a test could be used to chart performance improvements, ensure fitness to a prior level, or access different training programs.

A simple cardiac assessment test would also have value in the monitoring of individuals at risk for the development of heart failure. A host of comorbid conditions increase the risk of developing heart failure and include coronary artery disease, valvular heart disease, diabetes, dyslipidemia, metabolic syndrome, obesity, alcohol use, tobacco use, sleep apnea, chronic renal insufficiency, and hypertension. The consequence of heart failure on the individual patient is significant as are the medical expenditures. Heart failure represents a significant health care challenge in the US due to its high prevalence, morbidity, mortality, and treatment cost. The number of HF patients is increasing dramatically, from 5.1 million in 2012 to an estimated 8.0 million by 2030. These patients consume 34% of the total Medicare budget, an expected $67.7 B in 2030. Those patients at risk for developing heart failure could be proactively monitored. If an abnormal degradation of cardiac function were detected, more proactive management of the patient could be initiated with the goal of avoiding progress to heart failure.

A simple, non-invasive, and passive method and system for the assessment of cardiac function via a singular measurement or multiple measurements over time for determining the level of cardiac fitness relative to the population or changes in an individual's cardiac function would satisfy a well-defined need.

### SUMMARY OF THE INVENTION

Example embodiments of the present invention provide an apparatus for determining the cardiac fitness of a user, comprising: (a) a noninvasive sensor system, comprising one or more cardiovascular sensors configured to produce a signal that indicates a time of opening and closing of the user's aortic valve; (b) an initiation system, configured to detect an event indicating a cardiac fitness test is to be initiated; (c) a sensor control system responsive to the initiation system configured to operate the noninvasive sensor system at a first set of operational parameters to produce a first measurement signal that indicates the times of opening and closing of the user's aortic valve during two or more successive cardiac cycles; (d) a physiological assessment system configured to determine the presence of a basal physiological state from the first measurement signal based on (1) an interbeat time interval between successive openings of the user's aortic valve from each of two or more cardiac cycles, and (2) a variability between two or more interbeat time intervals, (e) a trigger system, responsive to the physiological assessment system; (f) a cardiac fitness assessment system responsive to the trigger system configured to activate when the trigger system indicates that a basal physiological state is detected and further configured to determine a first cardiac fitness score from the first measurement signal based on an ejection time interval between an opening and an immediately subsequent closing of the user's aortic valve; (g) a cardiac fitness reporting system configured to report the first cardiac fitness score.

In some embodiments, the sensor control system is responsive to the trigger system and is configured to operate the sensor system at a second set of operational parameters to produce a second measurement signal when the trigger system indicates that a basal physiological state is detected, and wherein the cardiac fitness assessment system is configured to determine a second cardiac fitness score from the second measurement signal. In some embodiments, the sensor system comprises optical emitters and detectors. In some embodiments, the noninvasive sensor system includes at least one of the following: electrocardiogram sensor, phonocardiogram sensor, seismocardiogram sensor, ballistocardiogram sensor, or echocardiogram sensor.

Example embodiments of the present invention provide an apparatus for determining the basal cardiac fitness of a user, comprising: (a) an optical measurement system comprising (i) one or more optical emitters configured to emit light toward a measurement region of the user and (ii) one or more detectors configured such that light reaches the detectors from the one or more emitters after the light from the emitters has interacted with the measurement region; (b) a sensor control system configured to operate the one or more emitters and the one or more detectors at a first set of operational parameters to detect changes in blood flow or blood volume to produce a first measurement signal that is indicative of opening and closing of the user's aortic valve; (c) a physiological assessment system configured to detect the presence of a basal physiological state from the first measurement signal based on a determination of (1) an interbeat time interval between successive openings of the user's aortic valve at each of two or more cardiac cycles, and (2) a variability between two or more interbeat time intervals; (d) a trigger system configured to respond to the presence of a basal physiological state as determined by the physiological assessment system; (e) a cardiac fitness assessment system responsive to the trigger system and configured to determine a first cardiac fitness score based on an interbeat interval from an aortic valve opening between successive openings of the user's aortic valve and a determination of ejection time from the first measurement signal based on the time interval between an opening and an immediately subsequent closing of the user's aortic valve; (f) a cardiac fitness reporting system configured to report the cardiac fitness score.

In some embodiments, the sensor control system is responsive to the trigger system and operates the sensor system at a second set of operational parameters to produce a second measurement signal when the trigger system indicates that a basal physiological state is detected, and wherein the cardiac fitness assessment system is configured to determine a second cardiac fitness score from the second measurement signal. In some embodiments, the optical measurement system includes a speckle plethysmography sensor. In some embodiments, the optical measurement system includes a photo plethysmography sensor.

Example embodiments of the present invention provide a method for determining a basal cardiac fitness of a user in an unstressed state, comprising: (a) providing a noninvasive sensor system configured to detect changes in blood volume or blood flow in a measurement region of the user, where the changes are indicative of opening and closing of the user's aortic valve; (b) acquiring a measurement signal from the noninvasive sensor system; (c) determining from the measurement signal an ejection time from an aortic valve opening until a successive aortic valve closing, and two or more interbeat intervals, where the interbeat interval is the time from an aortic valve opening until a successive aortic valve opening; (d) determining the presence of preload independence and the presence of cardiac vagal control based on the interbeat intervals; (e) if preload independence and cardiac vagal control are present, then determining a cardiac fitness score based on the ejection time; (f) reporting the cardiac fitness score.

In some embodiments, determining the presence of preload independence and the presence of cardiac vagal control comprises determining one or more measures of centrality and one or more measures of variability of two or more interbeat intervals. In some embodiments, determining the presence of preload independence and the presence of cardiac vagal control comprises comparing the measures of centrality and variability to historical values for the user. In some embodiments, determining the presence of preload independence comprises determining the presence of preload independence in the presence of a change in venous return to the heart. In some embodiments, determining the presence of preload independence comprises comparing determining a first interbeat interval, raising a leg of the user, determining a second interbeat interval, and comparing the first and second interbeat intervals.

Example embodiments of the present invention provide a method for determining a basal cardiac fitness of a user, comprising: (a) providing a noninvasive sensor configured to detect changes in blood volume or flow in a measurement region of the user; (b) providing a sensor control system configured to operate the noninvasive sensor at operational parameters to acquire a measurement signal; (c) providing a physiological assessment system configured to determine the presence of a basal physiological state from a measurement signal; (d) providing a trigger system configured to trigger the sensor control system to alter operational parameters if a basal physiological state is determined; (e) providing a cardiac fitness assessment system configured to determine a cardiac fitness score from a measurement signal; (f) using the sensor control system sensor to operate the noninvasive sensor at a first set of operational parameters to produce a first measurement signal; (g) using the physiological assessment system to determine the presence of a basal physiological state from the first measurement signal; (h) if a basal physiological state is determined, using the trigger system to trigger the sensor control system to alter operational parameters; (i) using the sensor control system sensor to operate the noninvasive sensor at a second set of operational parameters to produce a second measurement signal; (j) using the cardiac fitness assessment system configured to determine a cardiac fitness score from the second measurement signal; (k) reporting the cardiac fitness score.

In some embodiments, the physiological assessment system is a prediction model that maps systolic time interval information contained in the first measurement signal to the presence or absence of a basal physiological state. In some embodiments, the cardiac fitness assessment system is a prediction model that maps systolic time interval information contained in the second measurement signal to a cardiac fitness score. In some embodiments, the physiological assessment system uses a model that comprises multiple hierarchical layers. In some embodiments, the cardiac fitness assessment system uses a model that comprises multiple hierarchical layers.

Example embodiments of the present invention provide a method for determining a basal cardiac fitness of a user, comprising: (a) acquiring a first measurement signal from a noninvasive sensor configured to detect changes in blood volume or flow in a measurement region of the user, where changes contain systolic time interval information; (b) providing a physiological assessment system configured to analyze the measured systolic time interval information to determine the presence of a basal physiological state; (c) providing a trigger system configured to indicate the presence of a basal physiological state as determined by the physiological assessment system; (d) providing a cardiac fitness assessment system configured to analyze the measured systolic time interval information to determine a cardiac fitness score; (e) providing a cardiac fitness reporting system to provide the cardiac fitness score

Example embodiments of the present invention provide a method for determining a basal cardiac fitness of a user, comprising: (a) acquiring a first signal from a noninvasive sensor configured to detect changes in blood volume or flow in a measurement region of the user, where the measured signal contains systolic time interval information; (b) applying a basal physiological detection model to the measured signal to determine the presence of a basal physiological state; (c) if the presence of a basal physiological state is detected, applying a basal cardiac fitness model to the measured signal to determine a cardiac fitness score; (d) reporting the cardiac fitness score.

### BRIEF DESCRIPTION OF THE DRAWINGS

FIG. 1 illustrates systolic time interval measurements

FIG. 2 illustrates the relationship between several measurable signals and cardiac function.

FIG. 3 is a general illustration of the systems used in determining cardiac fitness

FIG. 4 is a second general illustration of the systems used in determining cardiac fitness

FIG. 5 illustrates an example method for determining cardiac fitness

FIG. 6 illustrates an additional example method for determining cardiac fitness

FIG. 7 Illustration of multi-step physiological assessment

FIG. 8 illustrates the use of the invention to access cardiac fitness over time

FIG. 9 the Frank-Starling curve and the location of preload independence

FIG. 10 illustrates other passive leg-raising maneuvers

FIG. 11 illustrates a coordinate system for the assessment of cardiac vagal control

FIG. 12 shows multiple sampling locations and example devices.

FIG. 13 illustrates the impact of resolution on the ability to detect aortic closure

FIG. 14 shows the effect of decreasing transmural pressure on pulse size.

FIG. 15 shows the effect of changes in hydrostatic pressure on pulse size.

FIG. 16 shows the time course of the aortic valve

FIG. 17 is a list of potential criteria, evaluations, and mitigations for ensuring accurate measurements

FIG. 18 is a flow chart for the determination of preload independence.

FIG. 19 is a flow chart for the determination of cardiac vagal control.

FIG. 20 is an example method for cardiac fitness testing in the medical clinic.

FIG. 21 is an example apparatus for clinic-based testing

FIG. 22 is an example of information flow associated with medical clinic testing

FIG. 23 is an example method for limited-duration testing in the home

FIG. 24 is an example apparatus for limited-duration home testing

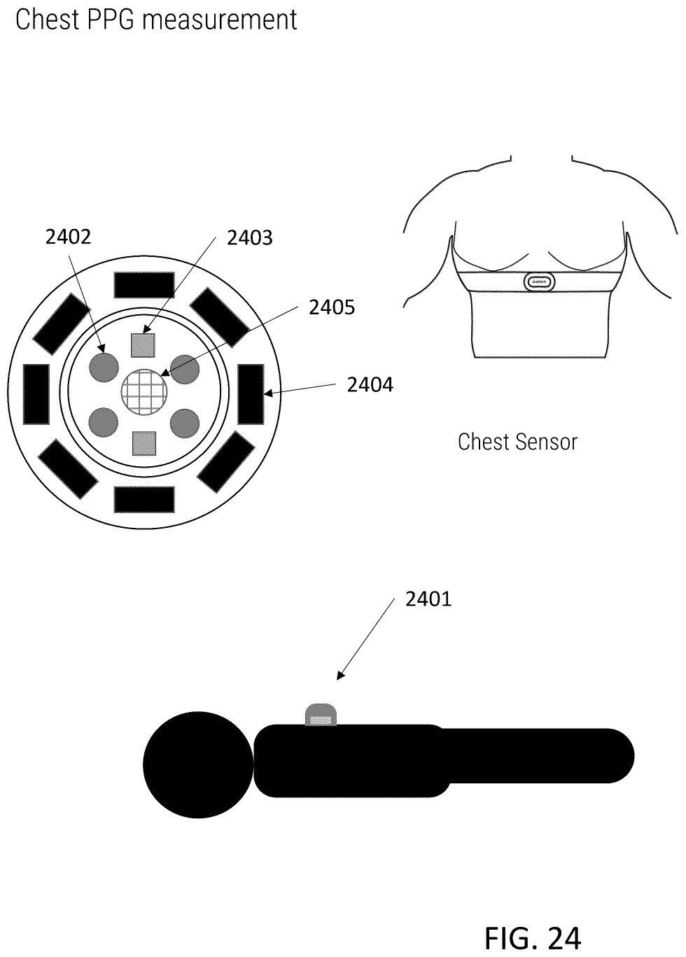

FIG. 25 is an example of information flow associated with limited-duration home testing

FIG. 26 is an example method for longer-term home testing

FIG. 27 is an illustration of criteria and a process flow for identifying optimal or extrema conditions

FIG. 28 is an example apparatus for longer-term home testing

FIG. 29 is an example of information flow associated with longer-term home testing

### DESCRIPTION OF INVENTION

Determining the heart's fundamental ability to pump blood is difficult and is typically done via a physical stress test. These stress tests are less than ideal as they are difficult to implement, require trained personnel to operate, and depend on other physiological systems. Embodiments of the present invention provide an easy-to-use test that is specific for basal cardiac function. Embodiments of the present invention require minimal training on the part of the user, can be done at home, and is not strongly affected by the capabilities of other physiological systems. The test determines basal cardiac fitness. Embodiments of the present invention identify a basal cardiac state by requiring the presence of specific physiological criteria: preload independence and cardiac vagal control. Preload independence creates a repeatable amount of myocardial muscle fiber stretch or tension before the start of ventricular contraction. Cardiac vagal control is an autonomic state when the vagus nerve alters heart rate with high responsivity, precision, and sensitivity. Cardiac vagal control occurs when the parasympathetic nervous system exerts greater control over cardiac function (heart rate and contractility) than the sympathetic nervous system, and sympathetic activation is low. Cardiac vagal control, as an autonomic state, can be inferred by using physiologically derived measures obtained noninvasively. In the presence of these appropriate physiological conditions, cardiac fitness is accessed by measuring cardiometric signals for the calculation of systolic time intervals. Systolic time intervals are temporal measurements influenced by cardiac performance and include the pre-ejection period (PEP) and left ventricular ejection time (LVET). The invention determines the individual's basal cardiac fitness based on the systolic time interval parameters or measured signals containing systolic time interval information obtained during a single measurement period meeting physiological criteria or from multiple measurements over time.

### Definitions

### General Terms

Measuring or measurement process, as used herein, refers to the process of obtaining a signal from a sensor.

A measurement signal or measured signal, as used herein, is the raw data or information obtained from a sensor system during a measurement process. Measurement signals are processed by analysis systems.

A parameter, as used herein, is a value that characterizes, summarizes, defines, or describes the properties of an entity. For example, a parameter may be calculated from a measurement signal to describe the properties of the signal. A parameter may also describe the properties of an individual (e.g., age, gender, weight, or the presence of a medical condition).

### Cardiac Function Parameters

Basal cardiac fitness, as used herein, is an assessment of cardiac fitness under repeatable and defined conditions that allow for demographic comparisons and comparisons over time.

Cardiac fitness score, as used herein, is the parameter representation of basal cardiac fitness generated by the invention. The cardiac fitness score is an assessment of an individual's cardiac fitness as measured in a defined and repeatable physiological state. The cardiac fitness score can be represented in different forms to aid users with interpretation. For example, the score can be provided as a measure compared to demographically matched individuals, as a comparison to prior values for the same individual, or as a result relative to the entire population. A descriptive statistical package could include trend lines and other graphs. The score can be presented as an absolute numerical score, a relative or scaled numerical score, or a percentage change from a prior measurement. A user-specific cardiac fitness score defines the cardiac progression of the individual and is especially useful in detecting deterioration in cardiac function.

### Signals and Parameters

Physiological signals, as used herein, define signals associated with maintaining or restoring homeostasis for life. The basic processes of life include organization, metabolism, responsiveness, movements, and reproduction. The signals may be measured from a variety of measurement systems that use optical, photonic, electrical, and seismic detection technologies. The resulting measurement signals can be used to calculate physiological parameters for the assessment of physiological status.

Physiological parameters, as used herein, refer broadly to those parameters of physiology with a focus on those parameters that influence cardiac function. Physiological parameters include but are not limited to blood pressure, body temperature, breathing rate, interbeat time interval, blood oxygen saturation, body position, Interbeat time interval variability, cardiorespiratory phase, and various electrophysiological signals associated with the operation of a human body.

Demographic parameters can include but are not limited to age, gender, height, and weight.

Health status measures can include but are not limited to medical history, diabetes status, and medications.

Measures of centrality, as used herein, aim to identify the midpoint in a data set through statistical means. Known measures of centrality are mean, median, and mode.

Measures of variability, as used herein, aim to measure variance as a summary statistic that represents the amount of dispersion in a dataset. Known measures of variability are range, interquartile range, standard deviation, variance, and frequency distribution.

Ancillary information, as used herein, defines additional information used in the measurement process to include demographic parameters, health status measures, and other additional information that allows a more accurate and meaningful cardiac assessment to be generated.

Cardiometric signals, as used herein, define a subset of physiological signals that are specific to heart performance. The term is translated from Latin as “measurement of heart performance.” The signals may be measured with various systems that use optical, photonic, vibrational, electrical, radar, and seismic detection technologies. The detected signals are directly associated with cardiac function or represent a secondary measure correlated with heart function. The resulting measurement signals can be used to determine systolic time intervals for the assessment of cardiovascular system performance and diagnostics to include prevention and therapy of cardiovascular system diseases.

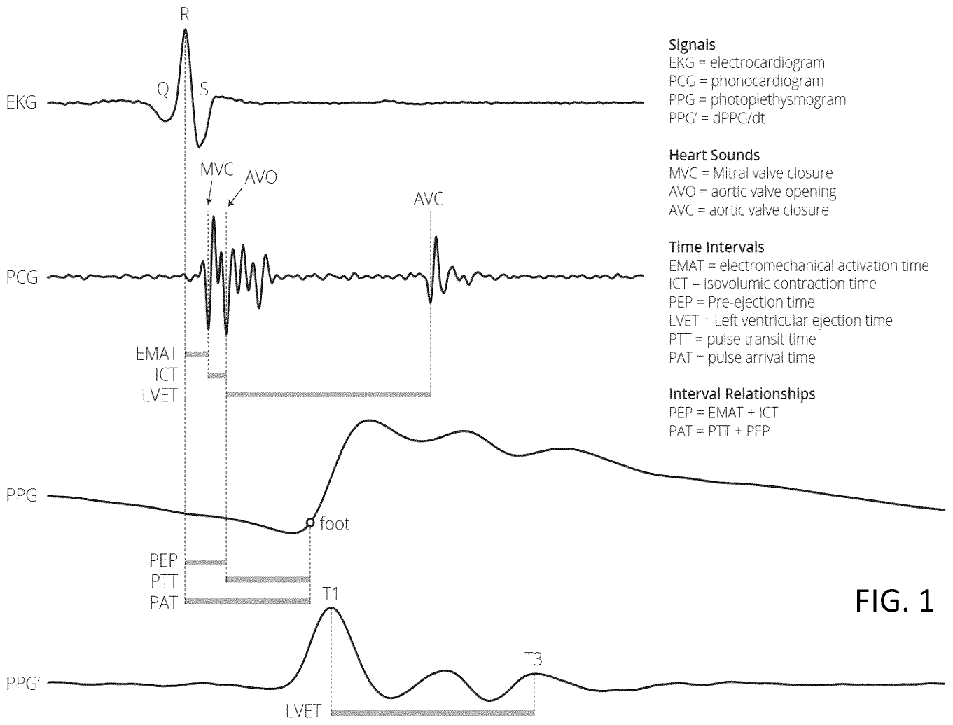

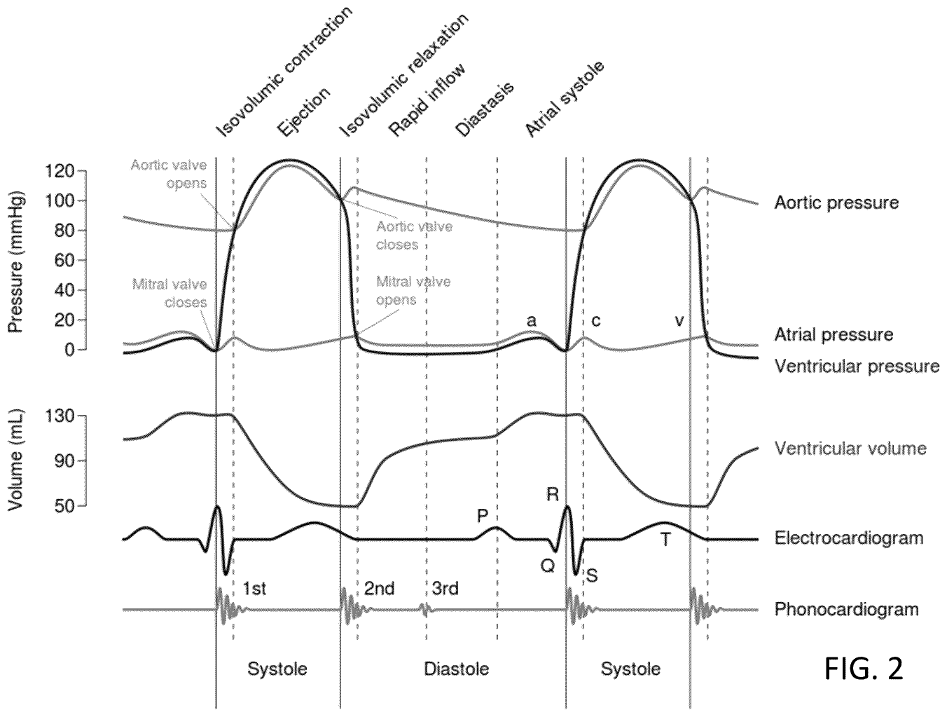

Systolic time intervals, as used herein, are one or more calculated or measured parameters that describe the temporal phases of the cardiac cycle. These parameters are influenced by left ventricular performance and can be used to quantify the strength of the heart's action or pumping capability. Cardiac-specific systolic time intervals include EMAT (electromechanical activation time), ICT (isovolumic contraction time), PEP (pre-ejection time), and LVET (left ventricular ejection time). Parameters associated with pulse transit times are PTT (pulse transit time) and PAT (pulse arrival time). FIG. 1 is an illustration of standard systolic time intervals. The relationship between cardiac function, volumes, pressures, and the time course of aortic valve status is illustrated in FIG. 2. The figure shows a time axis with pressure and volume relationship defined over the cardiac cycle with aortic and mitral valve functions illustrated. Left ventricular ejection time (LVET) is a parameter defined by the opening and closing of the aortic valve. Systolic time interval parameters can be used by prediction models for the assessment of physiological state and cardiac fitness.

Systolic time interval information, as used herein, is a measured signal that contains information related to the temporal descriptions of the phases of the cardiac cycle, is influenced by left ventricular performance, and can be used to quantify the strength of the heart's action or pumping capability. Measurement signals containing systolic time interval information include PPG, SPG, EKG, phonocardiogram, seismocardiogram, and ballistocardiography. Systolic time interval information can be used by matching models based on algorithms to include artificial intelligence, machine learning, and deep learning methods.

### Measurement and Sensors

Noninvasive sensors, as used herein, refers to a class of sensors that can be used outside the body and are sensitive to the opening and closing of the patient's aortic valve, physiological signals, and other cardiometric signals.

Cardiovascular sensor, as used herein, is any sensor that responds to and produces a signal that is indicative of activity of the heart, including as examples electrocardiogram, phonocardiogram, seismocardiogram, ballistocardiogram, echocardiogram, speckle plethysmogram, photo plethysmogram, radar plethysmography, vibration sensors, acoustic sensors, and optical sensors.

Electrocardiogram, as used herein, is a test that records the electrical activity of the heart. The measured signals can be used in both physiological assessments and the determination of cardiac fitness.

Phonocardiogram, as used herein, is a recording of the sounds made by the heart and are related to the mechanical activities of the heart. The measured signals can be used in both physiological assessments and the determination of cardiac fitness.

Seismocardiogram, as used herein, is a technique for recording and analyzing cardiac vibratory activity as a measure of cardiac contractile functions. The measured signals can be used in both physiological assessments and the determination of cardiac fitness.

Ballistocardiography, as used herein, is a technique for producing a graphical representation of the reaction of the body to cardiac ejection forces or the reaction of the body to the blood mass ejected by the heart with each contraction associated with arterial circulation. The measured signals can be used in both physiological assessments and the determination of cardiac fitness.

Vibrational and acoustic measures, as used herein, refers to those measurement technologies that are sensitive to the vibration generated by the heart and include phonocardiogram, seismocardiogram, ballistocardiography, or any other method that is sensitive to the vibrations or sound created by the heart.

Echocardiography, as used herein, is the use of ultrasound to investigate the action and functioning of the heart. The measured signals can be used in both physiological assessments and the determination of cardiac fitness.

Speckle plethysmography (SPG) is an optical measurement technology that measures changes in blood flow using laser speckle imaging and can be used in a transmission sampling mode and reflection sampling mode. The measured signals can be used to calculate both physiological and cardiometric parameters for both physiological assessments and the determination of cardiac fitness.

Photo plethysmography (PPG) is an optical measurement technology that measures changes in blood volume using changes in light absorption and can be used to measure blood volume in a transmission sampling mode and reflection sampling mode. The measured signals can be used to calculate both physiological and cardiometric parameters for both physiological assessments and the determination of cardiac fitness.

Radar plethysmography (RPG) is a noninvasive millimeter-wave, radar-based method for the accurate measurement of arterial pulse waveforms. Radar plethysmography can be utilized at any location on the body where a pulse creates a detectable movement of the skin or tissue. A common location is to use the system as a wrist-worn device that positions the radar near the radial artery without touching the skin, allowing for interrogation of the pulse at close range without perturbing the pulse waveform.

Optical sensors, as used herein, refers to any optically based system that can be used to capture signals related to physiological changes in blood volume, flow, or pressure in a measurement region of the individual, which changes are indicative of opening and closing of the individual's aortic valve. Additionally, optical sensors are sensitive to both physiological signals and cardiometric signals.

### Physiological States

An unstressed cardiac testing method, as used herein, defines test conditions that do not include activities that cause an increase in heart rate. Typical cardiac fitness tests are based on exercise or the response to some other stimulation resulting in a stress response of the heart. The invention does not use a stress response necessitating an increase in heart rate and is therefore referred to as an unstressed or resting-state test. Significant volitional movements during the cardiac assessment phase will result in an increased heart rate and are inconsistent with an unstressed state.

Basal physiological state, as used herein, is a resting state defined by the presence of cardiac vagal control and preload independence. An individual in a basal physiological state is resting, unstressed, and has a venous return at or near maximum capacity.

Cardiac vagal control, as used herein, defines as an autonomic state when the vagus nerve alters Interbeat time intervals with high responsivity, precision, and sensitivity. Cardiac vagal control occurs when sympathetic activation is low, and the parasympathetic nervous system exerts greater control over cardiac function (interbeat time interval and contractility) than the sympathetic nervous system.

Respiratory sinus arrhythmia (RSA), as used herein, refers to the presence of variation in the Interbeat time interval linked to the respiratory cycle. Specifically, the heart rate increases when breathing in and decreases when breathing out. Respiratory sinus arrhythmia is frequently used as a noninvasive method for investigating vagal tone in physiological and behavioral studies. Respiratory sinus arrhythmia is commonly observed during states of cardiac vagal control, but variations in the interbeat time interval during breathing can also be present under other physiological conditions, including dehydration or hypovolemia.

Preload independence, as used herein, defines a physiological state where the variations in cardiac filling pressures have minimal effect on stroke volume. Preload independence occurs during conditions of high venous return when the heart is filled at or around natural capacity. The location of the body in a supine position facilitates preload independence by increasing venous return.

An alteration in venous return, as used herein, refers to activities that change the filling pressure into the heart in a systematic fashion. Alterations in venous return can be accomplished but are not limited to intrathoracic pressure changes, changes in the total circulating volume, and alterations in the distribution or location of the circulating volume.

### System Operational Parameters

A measurement period is defined as a time duration over which measurements are made for the procurement of data for a single assessment of cardiac fitness. A measurement period can be of any length but is typically 24 hours or less.

An assessment period is the time duration over which an assessment of cardiac fitness is obtained and includes one or more measurement periods. An assessment period can be of any length but is typically on the order of several weeks to several months.

### System Components

A sensor system, as used herein, refers to software and hardware that measures the physical or electrical characteristics of cardiac function that enable a cardiac fitness measurement. The sensor system enables the sampling and recording or physiological and cardiometric signals. Capabilities of the sensor system can include but are not limited to sampling, conversion, filtering, amplification, optimization, signal quality assessment, processing, and recording. For illustrative purposes, a sensor system for optical measurements could be composed of an emitter, detector, power supply, and microcontroller containing one or more CPUs (processor cores) along with memory and programmable input/output peripherals and RAM.

An initiation system, as used herein, is the physical mechanism for initiating the start of cardiac fitness measurement. The initiation system may use an input for a sensor and/or a human user via a user interface. The system can be composed of a start button, a remote trigger system, and software that initiates a measurement after a fixed period or at a defined time of day. Current technology microcontrollers usually contain many general-purpose input/output pins (GPIO). GPIO pins are software configurable to either an input or an output state. When GPIO pins are configured to an input state, they are often used to read sensors or external signals.

A sensor control system, as used herein, refers to software and hardware that is designed to control one or more elements of the sensor system. In most implementations, the control system regulates the operation of the sensor system based on input or logic. The logic element is often implemented via a microcontroller with associated software representing the logic need for operation. The sensor control system can modify the operation of the sensor system to enable different data acquisition modes and initiate changes in the sensor used for signal acquisition..

A physiological assessment system, as used herein, refers to software and hardware that processes measured physiological signals, including ancillary information, if desired, to determine the patient's physiological state.

A trigger system, as used herein, refers to the software and hardware system that initiates the start of a function, activity, or operation based on defined criteria. The trigger system can signal the sensor control system to initiate an instruction sequence that changes the operation of the sensor system after a basal physiological state has been identified. Alternatively, the trigger system can interrupt or trigger a change in the processing objectives of the system after a basal physiological state has been determined. The trigger is a signal from one system to another system after defined criteria are satisfied that initiates a different processing or signal measurement process.

A cardiac fitness assessment system, as used herein, refers to software and hardware that processes cardiometric information, systolic time intervals, systolic time interval information and/or measured cardiometric sensor signals to determine a measure of cardiac fitness for the patient and generate a cardiac fitness score.

A cardiac fitness reporting system, as used herein, refers to hardware and software that provides information back to the designated person or designated system. The reporting system may use a designated graphical interface or may transmit information via Wi-Fi or Bluetooth to other display or presentation systems. The designated person could be the patient, medical staff, or provider. A designated system can include a screen display, printer, secondary data repository, or electronic medical record.

A sleep assessment system, as used herein, is used to determine the presence of sleep and can include the determination of the sleep stage.

### DETAILED DESCRIPTION OF THE INVENTION

The invention provides a reliable, convenient, non-invasive, and cost-effective assessment of cardiac fitness that is differentiated from existing testing methods. The test does not require the patient to engage in physical exertion and is not based on the stress response of the heart. Rather the test is conducted while the patient is in an unstressed condition and is based on the fundamental characteristics of left ventricular function. The cardiac assessment is based on systolic time intervals which enable assessment of left ventricular function but must be obtained under defined physiological conditions. The invention employs a physiological assessment to ensure measurement conditions are satisfied before making a cardiac assessment. The two physiological conditions define independent parameters impacting cardiac function. The first ensures a repeatable amount of filling pressure (preload) so the amount of myocardial muscle fiber stretch or tension before the start of ventricular contraction is repeatable. The second condition relates to nerve activation controlling contractility by the autonomic nervous system. The desired state is defined by a low sympathetic tone with high parasympathetic activation. Under these defined physiological conditions, calculated systolic time intervals or measured systolic time interval information can be used to access cardiac function and enable comparisons over time and between other patients.

During cardiac testing, the physiological assessment system determines if desired preload conditions and autonomic nervous system conditions are present by determining the presence of preload independence and cardiac vagal control. In the presence of preload independence and cardiac vagal control, cardiometric signals are acquired that contain temporal descriptions of the phases of the cardiac cycle. The temporal descriptions can contain information on the electrical activity of the heart as well as changes in blood volume, flow, or pressure of the individual, which changes are indicative of the opening and closing of the patient's aortic valve. The cardiometric signals are used to calculate systolic time intervals to determine a cardiac fitness score. Alternatively, the cardiometric data stream can be used to define a cardiac fitness score. The resulting cardiac fitness score can be used to determine the degree of cardiac dysfunction present and changes in cardiac performance.

The detection of cardiac function deterioration by the patient or by a physician is very difficult. For example, a patient presenting with difficulty walking stairs has multiple potential etiologies, including changes in weight, respiratory function, cardiac function, or musculoskeletal system changes. Calculated systolic time intervals or measured systolic time interval information under defined physiological conditions enables an assessment of basal cardiac fitness for accessing changes in cardiac performance. The failing left ventricle is characterized by a prolongation of the systolic pre-ejection period (PEP) and a reduction in the left ventricular ejection time (LVET), while total electromechanical systole remains relatively unaltered. The prolongation in the pre-ejection period is well correlated with the reduced stroke volume in heart failure. The reduction in left ventricular ejection time is also correlated with a reduction in stroke volume under defined physiological conditions. The compromised mechanical performance of the heart is responsible for these abnormal systolic time intervals and is associated with heart failure.

In the ailing heart, LVET decreases with disease progression due to ischemic heart disease, heart failure (HF), and hypertension patients. As left ventricular function deteriorates, the ability of the heart to produce contractile force is attenuated and the rate of left ventricular pressure rise (LV dP/dt) during the isovolumic contraction decreases, resulting in a prolongation of isovolumic contraction time or pre-ejection period. The ability of the ailing heart to maintain a high left ventricular pressure during the ejection period decreases, resulting in a reduction in LVET. Additionally, LVET will also shorten with left ventricular deterioration because of the prolonged isovolumic contraction time, which induces a delayed onset of ejection.

In the case of aortic stenosis, prolongation of LVET has been shown to be a measure of aortic stenosis. The physiology behind the elongation is based on analysis of the arterial pulse in patients with aortic stenosis that shows prolongation of the upstroke phase with decreased peak (pulsus parvus et tardus) in association with an overall pulse prolongation.

Calculated systolic time intervals or measurable systolic time interval information are both shown in FIG. 1, and include the relationship between cardiovascular function and a variety of measured signals, heart sounds, and time intervals. Specifically, the figure shows the relationships of the pre-ejection period and left ventricular ejection time with measurable elements of the cardiac cycle. Due to the interdependency of these measurements, cardiac fitness can be determined through other terms, a combination of terms, and other novel assessments.

### Cardiac Fitness System

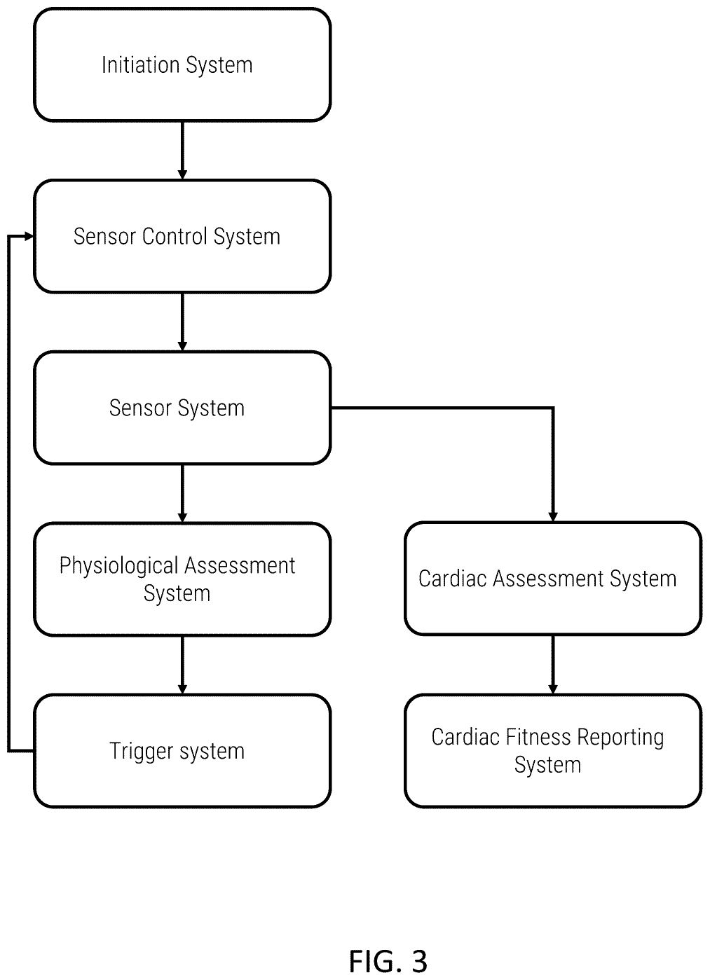

A cardiac fitness assessment system is composed of the following physical systems: initiation system, sensors control system, sensor system, physiological assessment system, trigger system, cardiac fitness assessment system, and cardiac fitness reporting system. FIG. 3 shows the systems and their points of interaction. As depicted, the trigger system is connected to the sensor control system and initiates a change in the operation of the sensor controls system after the detection of a basal physiological state. The cardiac fitness assessment system is connected to the sensor system as the sensor system has the measured signals needed for determining cardiac fitness.

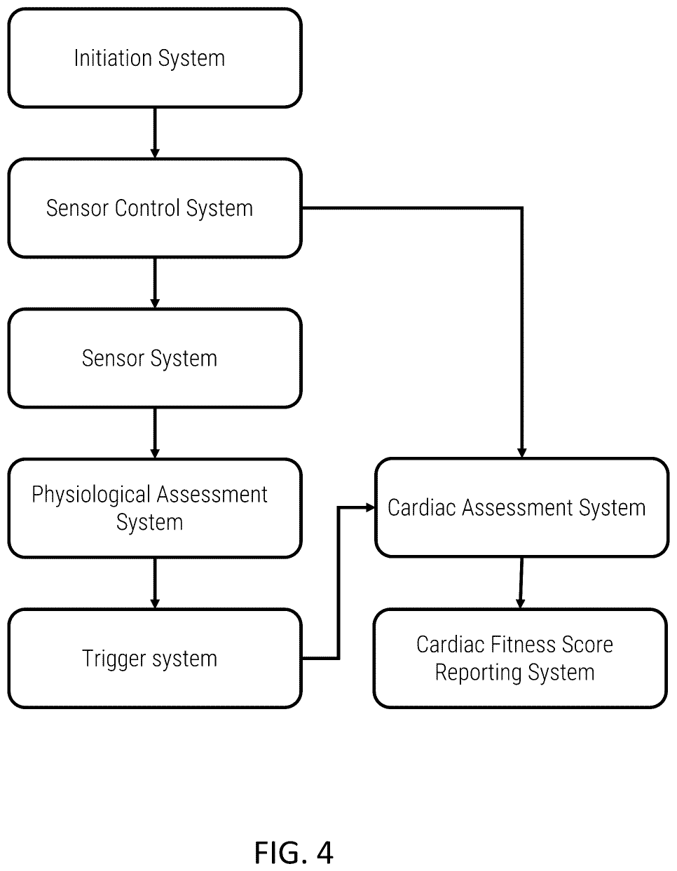

FIG. 4 shows a slightly different configuration where the trigger system is connected to the cardiac fitness assessment system. The trigger system initiates the hardware and software associated with the cardiac fitness assessment system. The cardiac fitness assessment system uses the trigger information as well as the measured signals to determine cardiac fitness.

### General Operation

The general operation of the system entails the determination of a basal physiological state by the physiological assessment system, followed by the determination of cardiac fitness by the cardiac fitness assessment system. The assessment of basal physiological status ensures that preload independence and cardiac vagal control are present so that the results appropriately reflect the patient's cardiac status versus alterations in volume status, myocardial muscle stretch, or sympathetic tone. The influence of these physiological variances is minimized by establishing a basal physiological state.

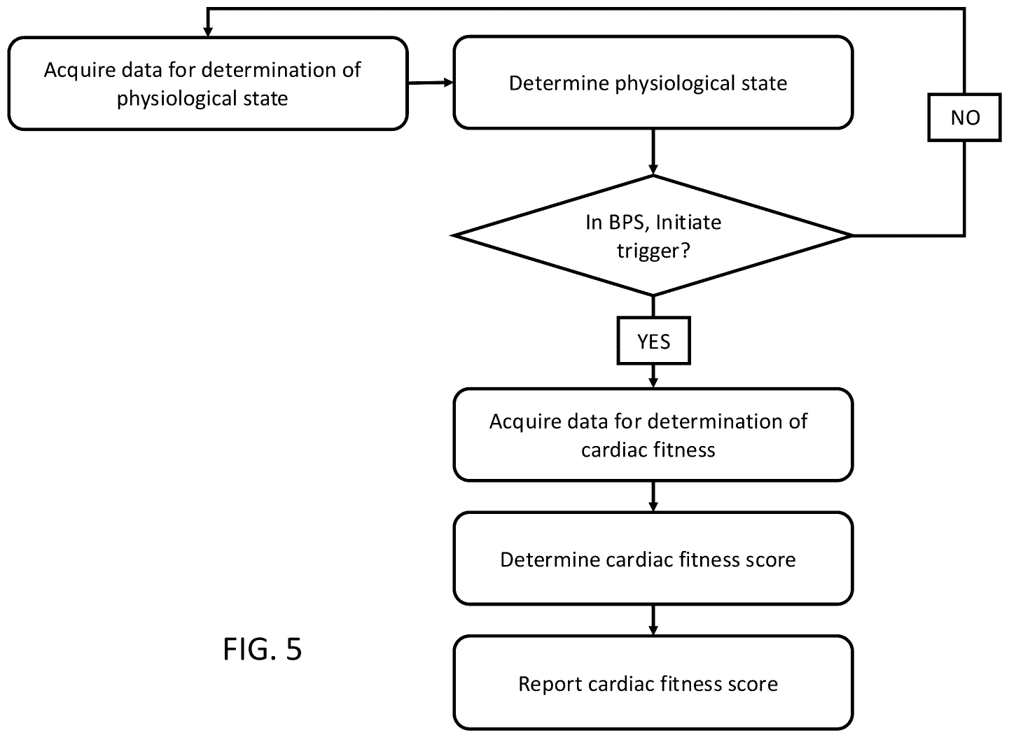

FIG. 5 is a general measurement protocol for determining cardiac fitness and communicates the need to determine a defined physiological state before the determination of cardiac fitness. The general process obtains physiological and cardiometric signals that are subsequently evaluated by specific assessment systems. Following an initiation event, the sensor control system and sensor system acquire data for the determination of the patient's physiological state. The measured physiological signals are processed by the hardware and software of the physiological assessment system to determine if a basal physiological state is present. If the desired basal physiological state is not satisfied, the system may request that the patient perform behaviors or assume body positions that would encourage a basal state, pause for a defined period, or simply acquire more data.

If a basal physiological state is present, then the hardware and software of the trigger system initiate an additional data acquisition by the sensor control system and sensor system for procurement of cardiometric signals that enable the determination of cardiac cycle events that may include the measurement of pre-ejection period and left ventricular ejection time. The trigger system defines an alteration in operation that only occurs following the detection of basal physiological state. The sensor control system may change the physical operating parameters of the sensors system, use additional sensors, or use different types of sensors. Potential changes in sensor operation can include sampling frequency and emitter intensity. The use of two sensor control modes can be advantageous with respect to battery management and data storage requirements.

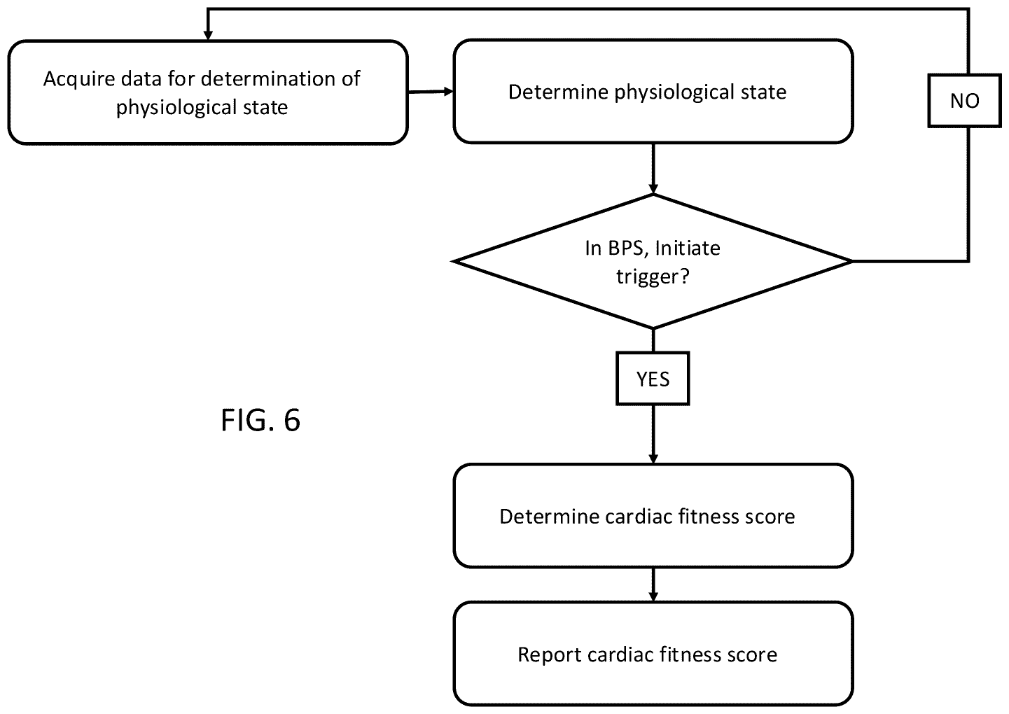

FIG. 6 shows an alteration in the operation of the system. Following an initiation event, the sensor control system and sensor system acquire data for the determination of the patient's physiological state. The measured physiological signals are processed by the hardware and software of the physiological assessment system to determine if a basal physiological state is present. If the desired basal physiological state is not satisfied, the system may request that the patient perform behaviors or assume body positions that would encourage a basal state, pause for a defined period, or simply acquire more data. However, if a basal physiological state is present, then the hardware and software of the trigger system will alter the processing sequence and initiates the determination of cardiac fitness based on the previously measured sensor signals.

FIG. 5 and FIG. 6 depict a sequential process. However, the activities can be conducted concurrently or in various orders. A critical element is to ensure that both the physiological assessment and the cardiac fitness determination are completed and consistent with measurement criteria before communicating a fitness result.

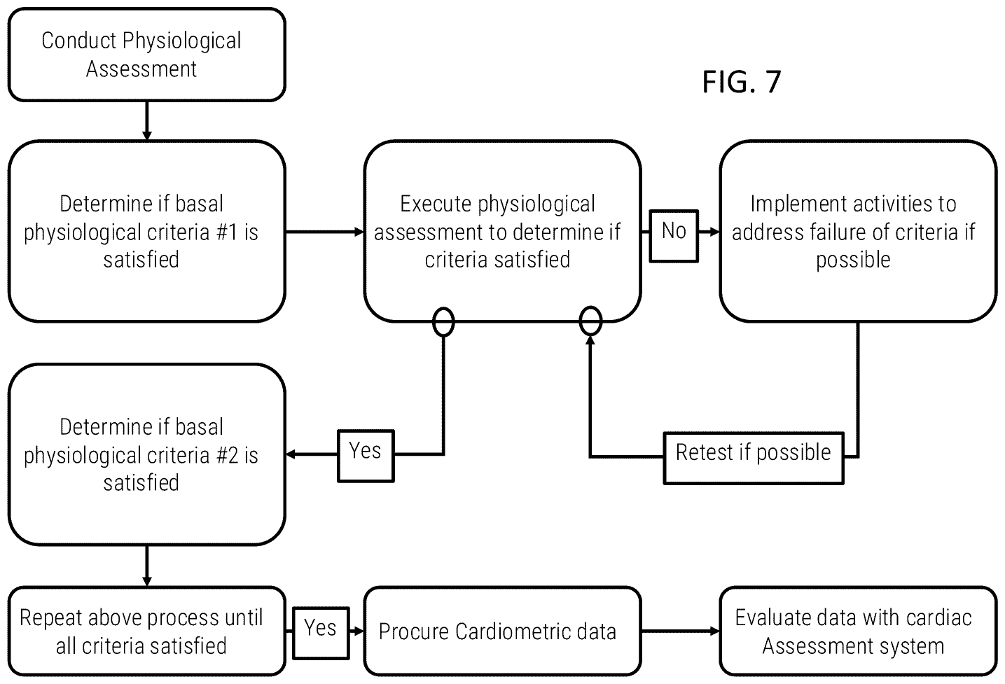

FIG. 7 illustrates the concept that the physiological assessment process can involve multiple steps. An evaluation process could start with evaluating the measured signals for the presence of a specific physiological state using defined criteria (preload independence). If the criteria have been satisfied, the evaluation moves to the next criteria until all criteria have been satisfied. If there is a failure to satisfy the criteria, mitigation activities are implemented to the extent possible. If retesting occurs, it is done until the criteria are satisfied. Depending on the test conditions, multiple criteria can be evaluated in a sequential or concurrent fashion. The use of multiple criteria ensures that the needed physiological state for an accurate measurement is obtained. After the criteria have been satisfied, cardiometric signals, either obtained during the physiological evaluation or obtained separately, are transferred to the cardiac assessment system for the determination of basal cardiac fitness.

In use, the evaluation criteria used may be based on prior patient information or based on the use of demographically matched information. Potential parameters for use in demographic matching and health status include but are not limited to age, gender, medical history, diabetes status, medications, height, and weight.

### Use of Information

The cardiac score can be used in a raw format, but additional value is created by comparing the score to prior information or other matched entities. For example, the cardiac fitness score of a 50-year-old marathon runner and a 50-year-old sedentary person could be very different. However, if their cardiac fitness scores have been stable over several years, neither may be progressing toward a heart failure condition. The evaluation system does this comparative assessment and generates a cardiac evaluation report that the patient and medical professional can use. The report is provided or transferred to the patient, medical professional, or systems of record, such as the electronic medical record. The report could also be transferred to other systems of record, for example, an application on the patient's phone. The resulting information is then used to create a cardiac maintenance or improvement plan.

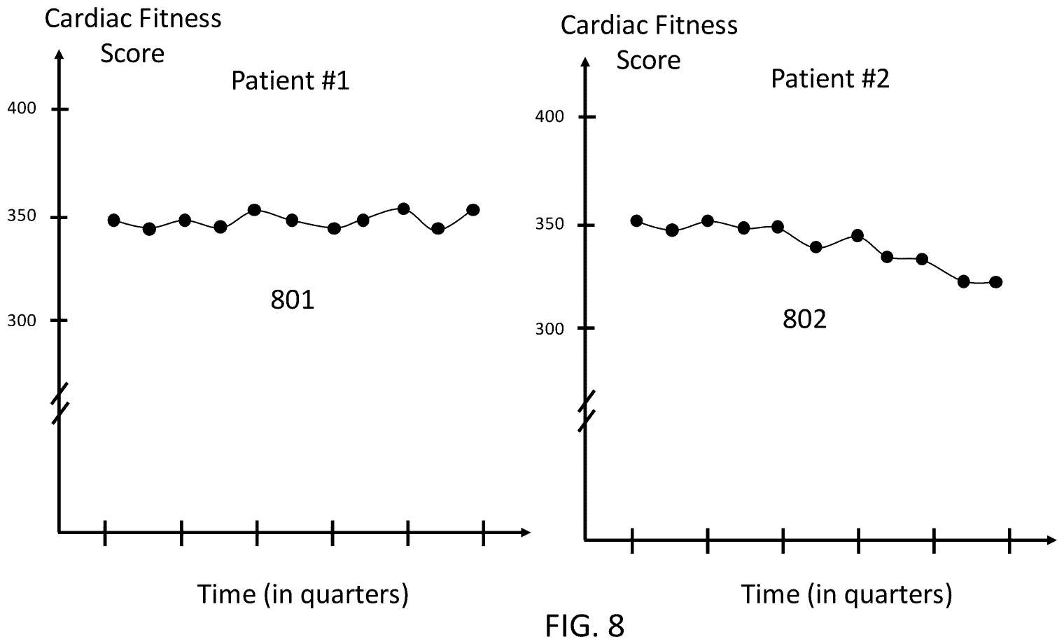

One feature of the invention is to provide basal cardiac fitness assessments over time so that interventions can be initiated if cardiac fitness is deteriorating. FIG. 8 is an illustration of the type of information generated and a proposed evaluation of cardiac fitness. The illustration shows cardiac fitness score on the Y-axis and time on the X-axis. The time axis duration is long enough to account for any hormonal changes, and the assessment period can be varied depending on the clinical condition. The cardiac fitness measurements of patient #1 show consistency over the measurement period, and there is no evidence of cardiac fitness degradation; see the plotted points (801). Patient number #2's overall trend line (802) shows a degradation in cardiac fitness. The following information can be used by the patient, provider, or other caregivers to inform changes that may reverse the undesired trend.

### Establishing Basal Cardiac Status

The accurate assessment of basal cardiac fitness requires that the heart is in a basal state during the measurement period. The state creates conditions with a repeatable degree of myocardial muscle fiber stretch or tension before the start of ventricular contraction and minimal sympathetic activation that affects cardiac contractility. Basal cardiac status is established when ventricular contraction is preload independent and under vagal control. The invention determines the presence of both conditions.

### Preload Independence

According to the Frank-Starling law, there is a positive relationship between preload and stroke volume; accordingly, the greater the ventricular preload (and therefore the degree of cardiac muscle stretch), the greater the stroke volume. However, this relationship, in the same way as in most physiological phenomena in the body, is not linear and traces a curve. Accordingly, once a level of preload value has been reached, further increments do not give rise to significant additional systolic volume elevations.

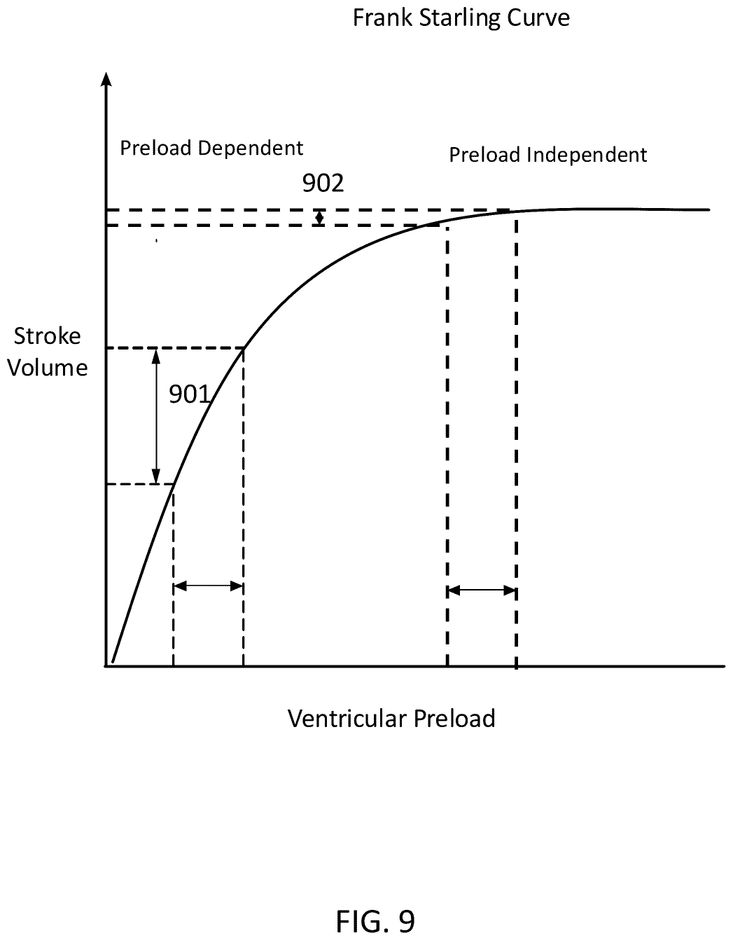

FIG. 9 illustrates the Frank-Starling curve with preload on the x-axis and stroke volume on the y-axis. The figure illustrates two zones of operation with dramatically different relationships between preload and stroke volume. The preload-dependent zone (901) denotes a zone of operation where minimum preload changes give rise to a marked increase in systolic volume, which is known as preload dependence. The preload independent zone (902) denotes a flatter zone of operations, where the ejection volume varies little with a change in preload, which is known as preload independence.

For basal cardiac fitness assessment, the test should be conducted during conditions of preload independence as it creates a repeatable amount of myocardial muscle fiber stretch or tension before the start of ventricular contraction. One method of achieving preload independence under typical physiological conditions is to have the patient in a supine position. A supine body position is typically associated with preload independence due to increased venous return. Movement from the standing position to the supine position results in the translocation of approximately 300 ml to 500 ml from the lower extremities towards the intrathoracic vessels and produces an increase in venous return and cardiac preload. However, the simple placement of the body in a supine condition does not guarantee preload independence. These conditions must be confirmed via additional assessments of interbeat time interval (IBI) and Interbeat time interval variability.

### Accessing Preload Independence Via Venous Return Change Evaluation

If multiple observations over time are not possible, preload independence can be accessed via activities that alter the venous return to the heart. The changes in venous return and corresponding preload should not result in a significant alteration in stroke volume if the patient's heart is operating on the plateau portion of the Frank-Starling curve, a condition of preload independence. There exist many mechanisms that can be used to alter venous return, including (1) intrathoracic pressure changes, (2) changes in the total circulating volume, and (3) alterations in the distribution or location of the volume. For basal cardiac assessment, any dynamic alteration of venous return should be conducted so the patient remains unstressed and time is allowed to obtain a physiological state of cardiac vagal control. For the determination of preload independence, the evaluation process involves a comparison of physiological parameters in the two venous return conditions. For example, suppose the condition of increased venous return increases stroke volume, as observed by an elongation of LVET or a decrease in heart rate. The comparison process can evaluate if the changes suggest a condition of preload dependence by comparing the physiological parameters to prior physiological measurements from the patient to demographically matched references or defined thresholds. Deterministic outputs, as well as probabilistic assessments, can be generated. Multiple methods for changing venous return are described below and may be used independently or in combination to determine preload independence.

Intrathoracic pressure changes can be used to alter venous return to assess preload independence. During inspiration, venous return increases as the intrathoracic pressure becomes more negative. The reduction in intrathoracic pressure draws more blood into the right atrium. Multiple types of breathing protocols can be used to alter venous return with subsequent assessment of LVET to determine preload independence. Various methods for changing intrathoracic pressure and, correspondingly, venous return are explained subsequently.

The Valsalva maneuver is an exaggerated exhalation, usually a sustained, forced exhalation against a closed glottis. During a maintained increase in intrathoracic pressure, venous return is interrupted, and stroke volume falls.

Resistance breathing is a general term that applies to any method that increases, decreases, or changes intrathoracic pressure over normal breathing and alters venous return. A resistance breathing test can include inhalation resistance breathing or exhalation resistance breathing, independently or in combination. The use of exhalation resistance breathing creates an increase in intrathoracic pressure while the use of inhalation resistance breathing creates decreased intrathoracic pressure. Additionally, the system can use different levels of resistance over the course of the protocol. Multiple methods of implementation exist for altering intrathoracic pressure above normal levels. The system can include the use of pressure threshold, flow-independent valves, air restriction mechanisms, and any mechanism that cause an increase in pressure during normal breathing. Additionally, the term resistance breathing covers the process of creating a change in intrathoracic pressure where little or no air movement occurs for a period of time. The creation of an occlusion pressure, either increased or decreased, is encompassed as part of the broad definition of resistance breathing. Resistance breathing is a method that can be used to change venous return to the heart and influences end-diastolic volume.

Paced breathing is a general term that applies to any method that alters breathing rate by defining rate and can include depth of breathing. Paced breathing is typically slow at a rate between 5 and 7 breaths per minute. With normal breathing, the rate is about 12 to 14 breaths a minute. Paced breathing can include defined changes in the rate as well as an asymmetric breathing profile, for example the exhale is 8 seconds while the inhale is 5 seconds.

Controlled breathing is the process of combining elements of paced breathing with resistance breathing. The “controlled” aspect is a system or method of breathing that dictates breathing rate and creates an intrathoracic pressure change that is greater than normal breathing. Examples of controlled breathing include but are not limited to a mini-Mueller inhale against resistance followed by a mini-Valsalva against resistance at a rate of 6 breaths per minute.

Changes in circulating volume by administration of IV fluids, often referred to as a volume challenge, can be used to alter venous return. Evaluation of the response to the administration of a given amount of volume (fluid challenge) can be used to access preload independence. Additionally, the execution of a hydration protocol can be initiated, and may include drinking fluids.

Body position changes are a simple and reliable method for altering the distribution of the circulating volume and changing preload, and evaluating preload dependence. Passively raising the legs to an angle of 45 degrees to the bed for at least 1 min is equivalent to a volume expansion of about 300 ml. The effect is only temporary so the maneuver is regarded as a test and can be repeated if necessary. The blood transfer from the lower extremities towards the intrathoracic vessels produces an increase in venous return and increased cardiac preload. Clinical studies have shown the usefulness of this maneuver in evaluating the response to volume expansion. These studies suggest that an increase of ≥10% in stroke volume during the first 60-90 s of the leg raising maneuver offers sensitivity and specificity performances of over 90% in predicting the capacity to increase stroke volume with the administration of fluids. For basal cardiac assessment, a defined increase in stroke volume as observed by an elongation of LVET or an increase in Interbeat time interval over the following minutes indicates that preload dependence is present, and the criteria for making a basal cardiac fitness measurement have not been satisfied.

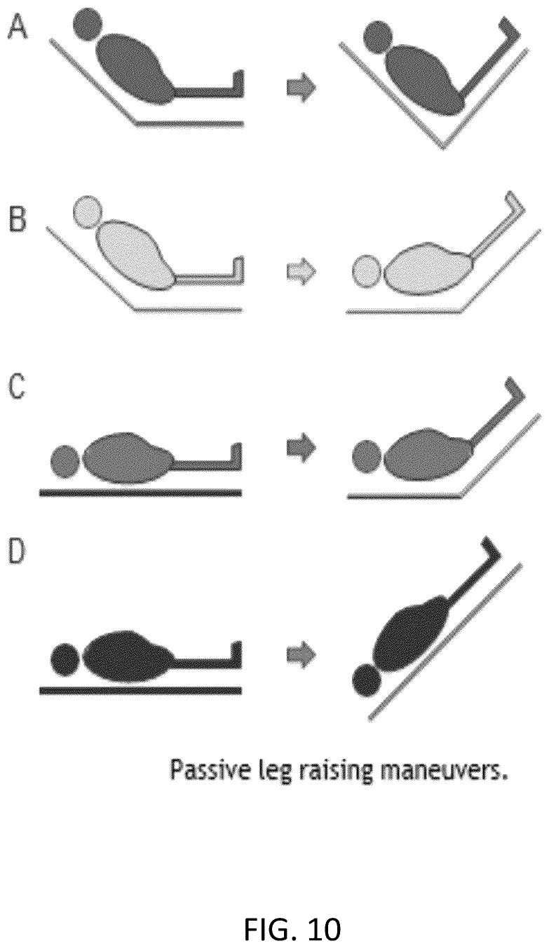

Other types of passive leg raising maneuver modalities can be used and are illustrated in FIG. 10. From the “semi-raised” position the legs can be elevated without lowering the trunk. This maneuver involves a lesser risk of aspiration and elevation of intracranial pressure (ICP) but generates less volume expansion since the splanchnic blood volume is not included. From the “semi-raised” position the legs can be elevated and the trunk can be lowered to zero degrees. From the supine position, the legs can be raised 45° without moving the trunk. The final maneuver involves rotation of the entire body. This maneuver causes significant changes in venous return but can result in some anxiety of the patient.

Any method or combination of methods that alters venous return can be used by the physiological assessment system for the assessment of preload independence. If the dynamic assessment of preload independence indicates preload dependence, the circulating volume of the patient can be altered by fluid consumption, or IV fluid administration and the patient retested.

For a reliable cardiac fitness assessment, the degree of change acceptable following a venous alteration will be defined and may be different based on the demographic and medical history of the patient, and the type of venous alteration used. In summary, preload independence can be accessed by examining one or multiple physiological parameters and determining if the percent change was less than a defined amount after the increase or reduction in preload. Candidate physiological parameters for preload assessment following venous changes include Interbeat time interval and LVET.

### Inferring Preload Independence Via Observational Parameters

During unstressed conditions, the body has a basal cardiac output requirement for the maintenance of metabolic functions. Cardiac output is the product of multiplying the stroke volume by heart rate, cardiac output=heart rate×stroke volume. In the absence of changes in sympathetic tone, heart rate and stroke volume have an inverse relationship. The body has a define cardiac output need, so if stroke volume decreases, then heart rate increases. Thus, heart rate can be used to access preload independence in an individual patient during the measurement period by conducting a comparative assessment and identifying a minimum or low heart rate. The low heart rate infers a high stroke volume which is linked to preload independence. The determination of a low heart rate for a given individual can occur via a comparative assessment based on historical heart rates from the user, other historical values, and other relevant comparison groups. Relevant comparison groups can include demographic matching, health status matching, medication matching, medical history match, or other relevant comparison groups.

For example, consider a measurement period where the patient sleeps in the supine position. The evaluation process for determining preload independence could include an assessment of body position and a comparative analysis of interbeat time intervals. The presence of a supine position can be determined in many ways, including direct measurements, inferred measurements or self-reported measurements. The effect of heart rate on ejection time interacts with body position. This physiological relationship was shown by Miyamoto et al, (Miyamoto, Y., Higuchi, J., Abe, Y., Hiura, T., Nakazono, Y., & Mikami, T. (1983). Dynamics of cardiac output and systolic time intervals in supine and upright exercise. Journal of Applied Physiology, 55(6), 1674-1681.).

After detection of a supine position, an interbeat time interval can be obtained, and a comparison assessment conducted. The comparison assessment could determine if the observed heart rate was a minimal heart rate or above average based on other measurements made during the assessment period, historical measurements from the patient, or relative to other matched patients. The comparative process can include demographic matching, health status matching, medication matching, medical history matching, or other relevant comparison groups. A higher heart rate could be associated with a lack of preload independence, so the criteria associated with the basal physiological status are not satisfied and no cardiac assessment is initiated. If the observed Interbeat time interval is consistent with preload independence, a trigger is initiated and a cardiac fitness assessment can be conducted.

In practice, nightly sleeping represents an appropriate measurement period as the patient is in the supine position and the duration of the measurement is several hours. Thus, one or more minimal or low heart rate observations can be used independently or in combination to create a cardiac fitness score for a given measurement period.

The length of the assessment period needs to account for the realities of obtaining measurement data. If the measurement is made in the clinic, then the assessment period will be defined by the availability of the patient and medical providers. If the determination of cardiac assessment can be made in the home over a longer period, it may be desirable to account for hormonal fluctuations that occur over a month. For example, the menstrual cycle influences both heart rate and heart rate variability. Over the menstrual cycle, the female body undergoes many hormonal changes that affect resting heart rate, heart rate variability, and body temperature. On average, heart rate increases between two and three beats per minute during fertile days preceding the monthly period. With an objective of measuring basal cardiac fitness, the desired objective would be to avoid periods of hormonally elevated heart rate or have the assessment period span the variance in hormonal levels.

### Cardiac Vagal Control

For the purpose of basal cardiac fitness, it is desired to have the contractility state of the muscle at a basal level with minimal influence of contractility or ionotropic agents. Specifically, autonomic sympathetic activation of the cardiac muscle should be minimal, as sympathetic activity impacts myocardial contractility far more than parasympathetic activation. General approaches to minimizing sympathetic activation include rest, calming environments, and sleeping. With respect to sleep, certain sleep cycles are considered more conducive to cardiac vagal control. Overall, sleep is considered a condition in which vagal activity is high and sympathetic activity is relatively quiescent. Within sleep stages non-rapid eye movement (non-REM) and REM sleep have lower levels of sympathetic tone relative to the phasic bursts of rapid eye movements characteristic of REM sleep reflecting sympathetic activation.

### Accessing Cardiac Vagal Control Via Physiological Measures

Cardiac vagal control is an autonomic state when the vagus nerve alters the interbeat time interval with high responsivity, precision, and sensitivity. Cardiac vagal control occurs when the parasympathetic nervous system exerts greater control over cardiac function (Interbeat time interval and contractility) than the sympathetic nervous system, and sympathetic activation is low. Cardiac vagal control, as an autonomic state, can be inferred by using physiologically derived measures obtained noninvasively. Cardiac vagal control can be inferred using one or more of the following measures of respiratory sinus arrhythmia, Interbeat time interval variability, vagal tone, the balance between the sympathetic and parasympathetic systems, a resting state, sleep state, low heart rate, and pulse variations.

Respiratory sinus arrhythmia is a physiological phenomenon where the heart rate accelerates during inspiration and slows down during expiration. Respiratory sinus arrhythmia is frequently used as a noninvasive method for investigating vagal tone is typically identified via electrocardiogramacording, PPG recording, SPG recording, and other noninvasive systems. Additionally, other methods have been developed that take advantage of the interactions between ventricular ejection and respiration. Interpretation of respiratory sinus arrhythmia measurements must be made with care, however, as several factors, including differences between individuals, can change the relationship between respiratory sinus arrhythmia and vagal tone. In practice, an estimate of respiratory sinus arrhythmia can be calculated by subtracting the shortest interbeat time interval during inspiration from the longest interbeat time interval during exhalation. RSA can be calculated via multiple methods, with some including breathing rate while others do not. The resulting RSA parameter is evaluated via a comparative assessment using historical values. The comparison assessment can determine if the observed RSA level is consistent with cardiac vagal control. The assessment of RSA can include other measurements made in an assessment period, historical measurements from the patient, or relative to other matched patients. The comparative process can include demographic matching, health status matching, medication matching, medical history matching, or other relevant comparison groups.

Heart rate variability (HRV) is the physiological phenomenon of variation in the time interval between heartbeats and is associated with respiratory sinus arrhythmia. It is measured by the variation in the beat-to-beat interval and has been described by more than 70 variables. HRV analysis can be performed in the time domain, in the frequency domain, and with non-linear indices.

Vagal tone can be accessed by examination of the high-frequency components of HRV. High-frequency heart rate variability is a frequency domain analysis typically between 0.15 and 0.40 Hz and is commonly associated with vagal tone.

The balance between the sympathetic and parasympathetic systems can be assessed by examination of the low frequencies/high-frequencies ratio, where low frequencies are typically defined as between 0.04 and 0.15 Hz.

A resting state is defined by the lack of significant volitional activities by the patient. Accelerometers in the measurement device are commonly used to assess the movement of the patient.

Low heart rate is evaluated using a naïve reference based on demographically matched values or prior observations with the patient.

The presence of sleep and the identification of the sleep stage can be based on heart rate variability. During normal sleep, the autonomic nervous system (ANS) modulates cardiovascular functions during sleep onset and the transition to different sleep stages. The analysis of heart rate variability (HRV) is a reliable tool to assess cardiovascular autonomic control as it can report physiological autonomic changes present during the wake-to-sleep transition, sleep onset, and different sleep stages: REM and NREM sleep. In addition to heart rate variability, heart rate, breathing rate, skin temperate, movement information and the time of day can be used to determine the presence of sleep. These parameters are used by the sleep assessment system to determine the presence of sleep as well as a sleep stage. The presence of sleep and a non-rapid eye movement sleep stage are associated with cardiac vagal control and can be used for the determination of cardiac vagal control.

The determination of cardiac vagal control can involve one or more of the above assessments, as well as additional metrics. These one or more metrics are evaluated by comparison to prior physiological measurements from the patient, the identification of physiological extrema, and the comparison to demographically matched references or defined thresholds. Deterministic outputs, as well as probabilistic assessments, can be generated.

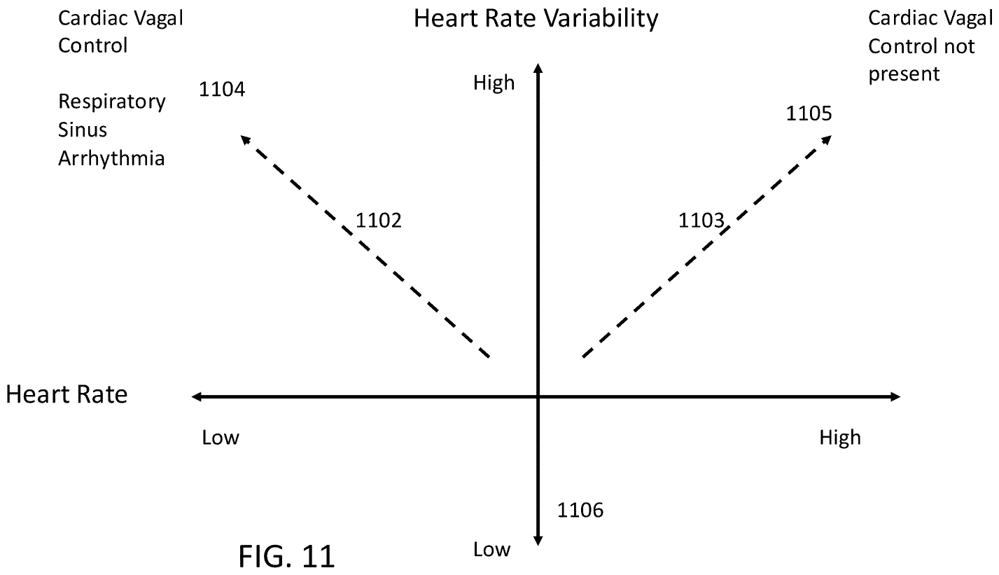

The presence of cardiac vagal control can be illustrated effectively in an XY coordinate system. FIG. 11 is an illustration demonstrating the relationship between several key measurement parameters and illustrates that heart rate variability cannot be used exclusively for the determination of cardiac vagal control. The X-axis is heart rate, and the Y-axis being heart rate variability. For basal cardiac fitness assessment, the desired location is in the upper left, with low heart rate, high heart rate variability, and the presence of respiratory sinus arrhythmia. The degree of respiratory sinus arrhythmia is illustrated by line (1102). A cardiac physiological condition satisfying the presence of respiratory sinus arrhythmia, high heart rate variability, and low heart rate is illustrated by numerical reference (1104) and would be consistent with cardiac vagal control. Numerical location (1105) illustrates a cardiac physiological state undesired for basal cardiac assessment because the physiological source of heart rate variability is not concurrently observed with a low heart rate. Locations with low heart rate variability, unless due to a disease condition or medications, as shown by numerical reference (1106) are not a desired physiological state for basal cardiac assessment and can be due to multiple etiologies, including sympathetic tone, exercise, catecholamines, or medications. The use of heart rate and heart rate variability creates a coordinate system for the determination of cardiac vagal control.

Cardiorespiratory phase synchronization is a recently developed metric that enables the assessment of autonomic status. Respiratory sinus arrhythmia is correlated with breathing and allows for the calculation of a phase relationship between heartbeat intervals and respiratory cycles. Recent advances in the field of nonlinear dynamics and statistical physics have led to the development of advanced phase-synchronization approaches that quantify cardiorespiratory coupling, specifically cardio-respiratory phase relationships (CRPS). In the context of cardiorespiratory coupling, phase-synchronization is defined as a consistent occurrence of heartbeats at the same relative phases within consecutive breathing cycles. The use of cardiorespiratory phase information provides an additional element for determining the presence of respiratory sinus arrhythmia and cardiac vagal control. The work of Bartsch et al. has demonstrated that the degree of cardiorespiratory phase synchronization in healthy subjects dramatically changes with sleep-stage transitions. Sleep-stage stratification demonstrated a lower CRPS during wake and REM when the sympathetic tone is dominant and a significantly higher synchronization during light sleep and deep sleep when the sympathetic tone is low. The changes indicate that sympathetic-parasympathetic balance strongly influences cardiorespiratory phase synchronization. (Bartsch, Ronny P., et al. “Phase transitions in physiologic coupling.” Proceedings of the National Academy of Sciences 109.26 (2012): 10181-10186.) As the determination of basal cardiac fitness is facilitated by the assessment of sympathetic-parasympathetic balance, the use of cardiorespiratory phase information provides additional information for the assessment of autonomic tone. The possible parameter for calculating of phase-synchronization patterns is the n: m synchronization percentage.

Breathing rate is also a parameter of interest and can be obtained via multiple modalities, including chest straps, PPG measurements, and breathing sensors. The respiratory rate has value in the determination of a restful state as well as the determination of cardiorespiratory phase relationships.

### Systolic Time Intervals

Systolic time intervals are noninvasive measurements that can be used for the assessment of left ventricular performance. The parameters are largely associated with left ventricular function and are related to the pumping characteristics of the heart. FIG. 1 shows the relationships between certain measured parameters associated with left ventricular function. Key time intervals include: electromechanical activation time (EMAT), isovolumic contraction time (ICT), Pre-ejection time (PEP), and left ventricular ejection time (LVET). The figure also illustrates interval relationships and heart sounds. The heart sounds are the bases of several measurement methods and can be used to derive valuable information.

### Pre-Ejection Period

The pre-ejection period (PEP) defines the time interval from the onset of ventricular depolarization to the opening of the aortic valve (i.e., the beginning of ventricular ejection). It comprises both the electromechanical activation time (EMAT) and isovolumic contraction time (ICT). The onset of ventricular depolarization is defined as the ECG R wave, as described above, and the opening of the aortic valve is determined from the first heart sound (S1) measured by PCG. Because aortic valve opening (AVO) lacks a distinct phonological signature in S1, we adopt the method of Paiva et al. and identify AVO using a Bayesian approach. Priors for AVO include (1) a local minimum in the PCG signal during S1, (2) large instantaneous amplitude as determined using the Hilbert Transform, and (3) a Gaussian distribution centered 30 ms after the closure of the mitral valve, which corresponds to the first negative deflection in S1. Paiva, R. P., et al. “Assessing PEP and LVET from heart sounds: algorithms and evaluation.” 2009 Annual International Conference of the IEEE Engineering in Medicine and Biology Society. IEEE, 2009. Note from FIG. 1 that PEP may also be defined as PEP=EMS−LVET, where EMS is electromechanical systole (the time interval from ventricular depolarization to the closure of the aortic valve) and LVET is the left ventricular ejection time. This approach is discussed below.

### Left Ventricular Ejection Time

The left ventricular ejection time (LVET) defines the duration of ventricular ejection, i.e., from the aortic valve opening (AVO) to the aortic valve closure (AVC). AVO can be determined from the first heart sound as defined above. AVC is defined as the start of the second heart sound (S2).

Alternatively, the LVET can be determined from PPG pulse waveforms measured at peripheral sites such as the finger or the ear. As shown by Quarry-Pigott et al., careful analysis of the derivative PPG waveform can identify transition points or peaks that correspond to the opening and closing of the aortic valve. Quarry-Pigott, Veronica, Raul Chirife, and David H. Spodick. “Ejection Time by Ear Densitogram and Its Derivative.” Circulation 48.2 (1973): 239-246. In one approach, shown in FIG. 1, LVET is defined as the interval between the first and third peaks in the first derivative of the PPG waveform. In a second approach, LVET is defined as the interval between the first and third peaks in the third derivative of the PPF waveform. When LVET can be determined from the PPG, PEP can be computed as PEP=EMS−LVET, where EMS defines the time interval from the ECG R wave to the second heart sound.

LVET as a cardiac fitness measure requires careful attention to parameters that transiently alter LVET but are not indicative of true changes in cardiac function. For example, LVET is impacted by blood pressure as demonstrated by the work of Scalzi et al., De Scalzi M, De Leonardis V, Citi S, Cinelli P. Relationship between systolic time intervals and arterial blood pressure. Clin Cardiol. 1986 November;9(11):545-9. doi: 10.1002/clc.4960091104. PMID: 3802602. The authors “suggest to consider the relation of STI to blood pressure to provide regression equations to best appreciate and use STI.” Thus, obtaining the same level of blood pressure, which is influenced by sympathetic tone, is an important consideration. Transient influences on LVET include increases in heart rate and blood pressure. Both shorten LVET due to changes in sympathetic tone versus a true change in cardiac fitness or pump capability. Thus, the determination of a basal physiological state becomes a necessity when accessing cardiac fitness via the use of LVET.

### Cardiorespiratory Phase Synchronization

Because the cardiorespiratory phase synchronization measurements represent a phase relationship, there exists the opportunity to use cardiorespiratory phase synchronization as an independent or additional assessment of cardiac fitness. Bartsch et al. observed a pronounced decrease in cardiorespiratory phase synchronization in subjects after a myocardial infarct. Thus, in conditions of cardiac compromise, the degree of cardiorespiratory phase synchronization decreases. Although cardiorespiratory phase synchronization has an age dependency, it does support between and within-subject comparison. As cardiorespiratory phase synchronization is based on the relationship between breathing and the initiation of heart contraction, it creates the opportunity for additional information on cardiac fitness. The resulting information can be used independently or in combination with cardiometric signals for the assessment of cardiac fitness.

### Other Cardiometric Parameters

Pulse amplitude describes the size of the pulse waveform as detected with the optical systems. Pulse amplitude can be computed as pulse height, from the foot of the waveform to the peak, or as area under the curve (AUC), the area under the PPG waveform from foot-to-foot. In our experience, AUC can be a more robust measure of pulse amplitude. Over long time periods, changes in pulse amplitude can reflect many factors, including vascular tone, body position, and PPG sensor attachment. However, over short time periods (minutes) where body position and vascular tone are relatively constant, the primary factor affecting pulse amplitude is pulse pressure, which is directly influenced by cardiac function.

The pulse contour describes the shape of the pulse waveform. The peripheral pulse waveform reflects a summation of the primary wave and secondary waves that arise from various reflections in the vascular tree. Changes in volume status, cardiac function, and stroke volume impact the size of reflected waves relative to the primary wave. Because the pulse waveform varies in amplitude, frequency, and shape, quantification methods vary and include frequency analysis, wavelet transformation, various decomposition methods, and curve fitting. An example curve fitting approach uses a mixture of Gaussians which capture the relative timing and amplitude of primary and reflected pulse waves. The resulting model parameters can be used to assess cardiac function.

### Factors Influencing Comparisons Between Individuals

Analyzing measured physiological parameters and applying the principles of FIG. 11 to the process should involve consideration of demographic, health status, and other external influences. An 85-year-old patient's cardiometric parameters may indicate decreased cardiac fitness compared to a general average or a 50-year-old patient but entirely normal compared to other 85-year-old individuals.

### Demographic Influences

Systolic time intervals are influenced by age and heart rate. Alhakak et al. state that, “There is a correlation between LVET and age, and LVET prolongs with aging. LVET is also influenced by sex, and it has been demonstrated that LVET at all heart rates was significantly longer in females than in males. LVET should be corrected for heart rate (HR) using sex-specific resting regression equations (for male: LVETI=1.7×HR+LVET, and for female: LVETI=1.6×HR+LVET).” (Alhakak, Alia S., et al. “The significance of left ventricular ejection time in heart failure with reduced ejection fraction.” European Journal of Heart Failure 23.4 (2021): 541-551. Additionally, the work of Hassan et al. includes corrections for systolic time intervals with respect to age, gender, and heart rate, (Hassan, S., and P. Turner. “Systolic time intervals: a review of the method in the non-invasive investigation of cardiac function in health, disease and clinical pharmacology.” Postgraduate medical journal 59.693 (1983): 423-434.) Thus, when evaluating systolic time interval information, gender, age, and heart rate differences may need to be considered. For example, when providing a cardiac fitness score and a comparative result, the comparison may be more exact if the match comparison is to a “matched” individual of similar age, gender, physical fitness, and heart rate. Additionally, LVETI or related calculations that minimize the influence of heart rate may be used as comparison metrics.

Heart rate variability will vary across patients as a function of age and disease state. Age is one of the strongest factors that influence heart rate variability values. Lower heart rate variability generally indicates an increased biological age (older). Higher heart rate variability is correlated with increased fitness, health, and youthfulness. These studies, with demographic information, create the basis for the appropriate assessment of HRV. (Garavaglia, Leopoldo, et al. “The effect of age on the heart rate variability of healthy subjects.” Plos one 16.10 (2021): e0255894. and Natarajan, Aravind, et al. “Heart rate variability with photoplethysmography in 8 million individuals: a cross-sectional study.” The Lancet Digital Health 2.12 (2020): e650-e657.) In summary, the interpretation of systolic time intervals should account for demographic influences.

### Health Status Influences

Type 2 diabetes mellitus is associated with a decrease in heart rate variability. The systematic review of the literature by Benichou, et. al. concluded that type II diabetes is associated with an overall decrease in the HRV. The deleterious effects of altered glucose metabolism lead to cardiac autonomic neuropathy and result in decreased heart rate variability. (Benichou, Thomas, et al. “Heart rate variability in type 2 diabetes mellitus: A systematic review and meta-analysis.” PloS one 13.4 (2018): e0195166). Thus, when evaluating patients with diabetes or other disease conditions, the thresholds to heart rate variability and the determination of repository cardiac arrhythmias must be adjusted for the medical conditions of the patient.

### Other External Influences

Conditions of elevated heart rate can occur via multiple mechanisms, including high sympathetic tone, dehydration, and diminished preload resulting in preload dependence. Additionally, some patients have “white coat syndrome.” White coat syndrome occurs is when a patient develops high blood pressure and tachycardia when around doctors.

Elevation in heart rate can occur due to sympathetic tone, exercise, catecholamines, caffeine, or medications. Thus, determining the physiological state, especially in the clinic, can require a careful review so the root cause of the physiological assessment failure can be identified.

### Signal Measurement

### Sampling Locations

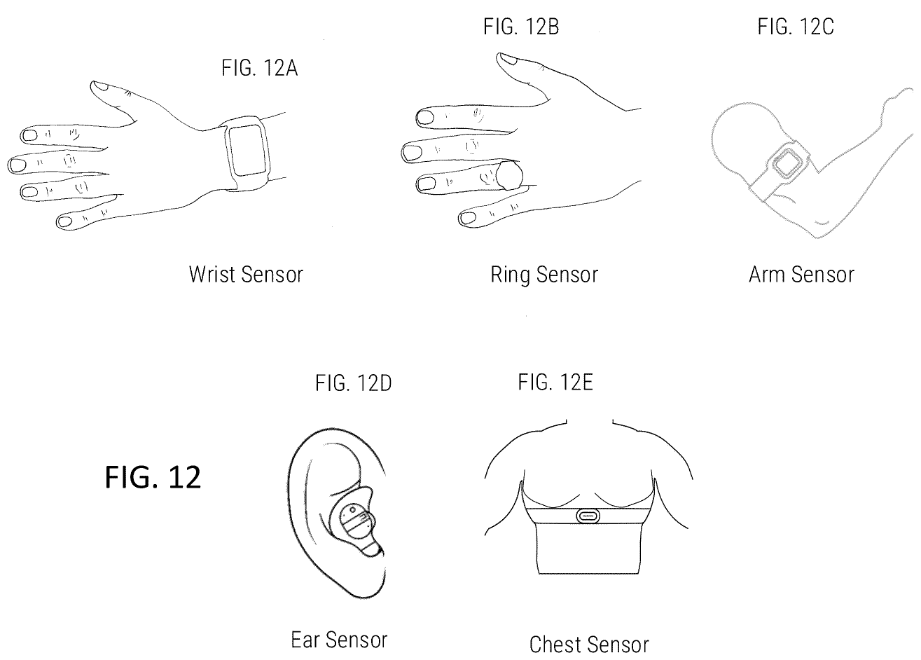

FIG. 12 illustrates several measurement locations where such sensors can be used to create data streams containing information on aortic valve opening and closing without interfering with the activities of daily living. The sensors can be based on optical, photonic, electrical, and seismic detection technologies.

### Measurement Instrumentation

Electrocardiogram (EKG) measurements are an essential element in the calculation of several time intervals including PEP. The wearable sensors as shown in FIG. 12 can include both optical sensors and EKG sensing capabilities.

Left ventricular ejection time (LVET), based on the time from the aortic valve opening (AVO) to the aortic valve closure (AVC), is an important systolic time interval for the determination of basal cardiac fitness. There are several sensor technologies capable of sensing aortic valve opening and closing. However, the ability to reliably detect aortic closure in a noninvasive and wearable device presents challenges that are specifically addressed by the current invention. A brief overview of sensing technologies is provided, followed by an in-depth discussion of some innovative elements of embodiments of the present invention that facilitate reliable aortic closure determination.

In medical settings, aortic valve closure is frequently determined from a central artery pressure waveform, as measured by Doppler ultrasound or invasive catheterization. The closure of the valve produces a downward notch in the aortic blood pressure, known as the incisura, due to a brief backflow of blood. The incisura is readily detected with ultrasound and catheterization; however, such measurement systems are inconvenient and inconsistent with simple clinic testing or self-testing testing in the home.

Optical sensors measuring changes in blood volume, commonly referred to as photoplethysmography (PPG) sensors, have the potential to measure aortic valve closure and are significantly more amenable to use in wearable devices. PPG sensors can be used on various locations on the body, including one or more fingers, one or more ears, and one or more wrists, chest, or forehead. PPG devices can also include image-based systems with spatial resolution over one or more dimensions.

Speckleplethysmography (SPG) is an optical signal that measures changes in blood flow using laser speckle imaging. Like PPG, it can be measured from the locations shown in FIG. 12 and processed in real time.

Methods such as laser Doppler flowmetry, tonometry, pulse transduction, and impedance cardiography (the measurement of electrical conductivity of the thorax) that are sensitive to changes in volume, flow, or pressure related to the cardiac cycle can also be used to acquire measurement signals indicative of aortic valve closure.

An alternative group of methods sensitive to the vibrations associated with the movement of the aortic valve includes phonocardiography, ballistocardiography, and seismocardiography. Phonocardiography (PCG) is a method of detecting the sounds produced by the heart and blood flow. Similar to auscultation, PCG is most commonly measured noninvasively from the chest with a microphone. Ballistocardiography (BCG) and seismocardiography (SCG) are both methods for studying the mechanical vibrations that are produced by the cardiac cycle. BCG is a method where the cardiac reaction forces acting on the body are measured. SCG, on the other hand, is a method where the local vibrations of the precordium (the region of the thorax immediately in front of the heart) are measured.

The preceding examples do not comprise an exhaustive list of technologies that can sense physiological changes associated with the opening and closing of the aortic valve but illustrate the variety of methods that have the potential to be used in the current invention.

### Optimization of Measured Parameters

The systems shown in FIG. 12 may resemble wearable devices currently available and designed for other purposes, but such “off-the-shelf” sensors cannot be used to reliably determine aortic valve closure. As an example, numerous currently available wearable PPG systems are designed to determine heart rate or heart rate variability. This determination requires only the measurement of signals or events associated with aortic valve opening. At the peripheral measurement site, the aortic valve opening manifests as a rapid increase in blood volume corresponding to the arrival of the pulse. Conventional wearable PPG heart rate monitors often use frequency or spectral analysis of the PPG signal to identify periodic changes in the PPG signal consistent

Thus, devices designed to measure heart rate or heart rate variability are not suitable for the reliable determination of aortic valve closure. In peripheral pulse waveforms, the signal associated with aortic valve closure is 50 to 100 times smaller than the signal associated with aortic valve opening. Accurate detection of aortic closure with a wearable device requires a carefully considered measurement system that incorporates physical and operational features distinct from those conventionally used to detect other physiological measures. In addition to a signal with aortic valve closure with significant signal to noise, the system for determining aortic value closure must have the hardware and software that enables the detection of this much smaller signal. The following sections detail these physical and operational features, with some details and examples specific to optical sensing technologies. One of skill in the art will recognize that many of the same principles can be used with alternative measurement technologies.

### Sampling Resolution

The ability to assess cardiac fitness at a level useful to the user requires high resolution of the change in blood volume, flow, or pressure in both the temporal domain and the signal amplitude domain. In the temporal domain, a sampling rate near or above 100 Hz facilitates determination of the events of aortic valve opening and closing to within 10 ms. Lower sampling rates can increase the error in ejection time calculation and hence subsequent cardiac fitness assessment. In the signal amplitude domain, amplitude resolution should be sufficient to resolve the changes associated with aortic closing, which are on the order of 1% of the magnitude of changes related to aortic valve opening. In embodiments where acquired measurement signals are digitized through an analog-to-digital converter, the bit-depth of the system should be sufficiently high such that signals related to the aortic valve closure are not lost with discretization.

In optical systems, the amplitude of signals associated with aortic valve closure can be enhanced by increasing the intensity or brightness of light used, provided that detectors and other aspects of the data acquisition system are not saturated. Light intensity can be increased with increased LED drive current or by increasing the number of LEDs in use, or both. Signal amplitude can also be increased by configuring additional operational parameters of the optical system, such as the integration time (length of time that photons are acquired at the detector). In wearable devices that are intended to be worn for prolonged periods battery life is always a concern. Because LED activation can produce a significant drain on batteries, overall LED intensity and duration of use can be considered prudently and used only as needed.

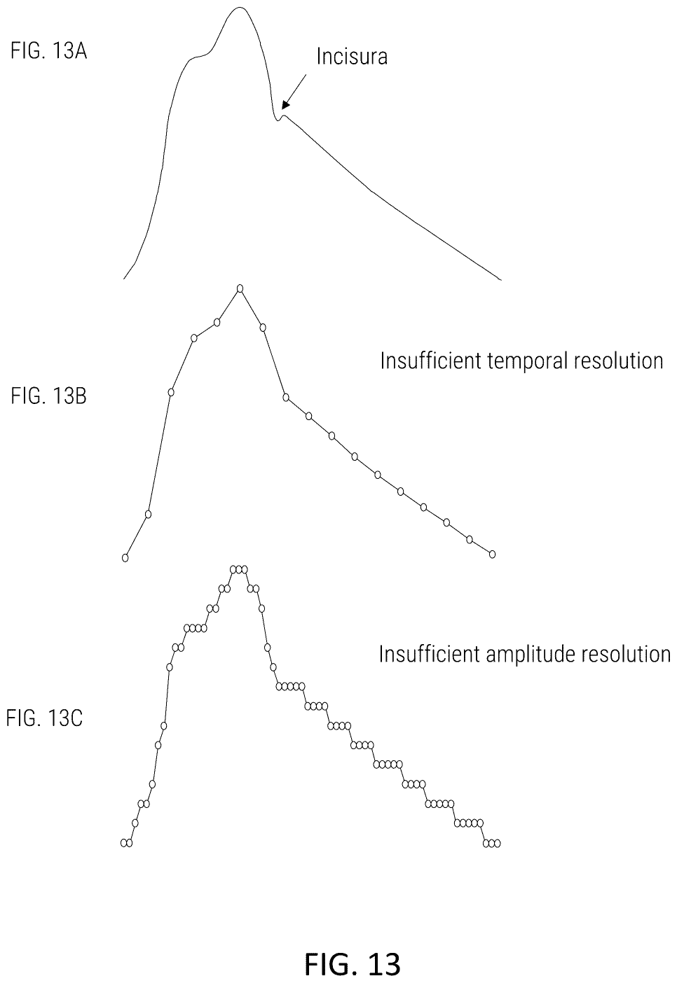

FIG. 13 demonstrates the effects of insufficient resolution on determination of aortic valve closure. FIG. 13A shows the pressure trace of a cardiac pulse sampled with high resolution in both domains. Aortic valve closure is determined from the incisura in the pressure wave. When the pulse is sampled with low temporal resolution of 16 Hz in FIG. 13B, the ability to determine the timing of the incisura is significantly degraded. A sampling rate of 16 Hz is common for heart rate determination in wearable devices but is insufficient for aortic valve closure determination. In FIG. 13C, the temporal resolution is improved but the resolution of the amplitude has been strongly degraded due to discretization. Here again, the precise timing of the aortic valve closure is difficult to discern. Thus, embodiments of the invention comprise a measurement system with the resolution in both the time and signal amplitude dimensions to enable the detection of the aortic valve closure.

### Sampled Vessels

For measurements of pressure, volume, or flow, the incisura signal associated with aortic valve closure will be largest at more proximal arterial segments and will dissipate along the vasculature tree. The signal will be more apparent in larger tri-layered vessels such as arteries and arterioles than in the largely inelastic capillaries.

The physical configuration of light emitters and detectors in an optical system also plays an important role in determining the optical path length and the type of vessels that are sampled. When the emitters and detectors are placed in close proximity (e.g., separated by <5 mm) the detected photons are more likely to have interacted primarily with superficial vessels in the capillary bed. When the detector is at greater separation from the emitters, the photons that reach the detector are more likely to have interacted with deeper tissue containing more proximal arterial segments. Because shorter wavelengths of light in the visible range are so strongly absorbed by tissue, emitters and detectors must be in relatively close proximity to enable sufficient photon detection. However, longer wavelengths in the red and near-infrared range can be used when emitters and detectors are physically separated by more than 10 mm, supporting optical paths where the majority of photos interact with artery and arteriole segments. To further encourage interaction with such vascular segments, emitters and detectors can be arranged such that the optical path traverses known anatomical locations of arteries. For example, in the fingers, the prominent palmar digital arteries run longitudinally along the sides of fingers, close to the volar surface of the hand. Therefore, more volar (ventral) placement of emitters and detectors can be advantageous to sample the arteries.

Notably, maximization of SNR related to aortic valve closure might not be equivalent to maximizing SNR for aortic valve opening. Because green light is so strongly absorbed by blood, the magnitude of the pulsatile signal associated with aortic valve opening can be significantly larger than the signal obtained with longer wavelengths. In addition, green light sensors are less influenced by venous compartments due to their shallow penetration depths, reducing sensitivity to some motion-related artifacts. The result is that for conventional wearable systems measuring heart rate and heart rate variability, green light can be optimal. This is taught, for example, by Maeda et al (Maeda, Y., Sekine, M., & Tamura, T. (2011). The advantages of wearable green reflected photoplethysmography. Journal of Medical Systems, 35(5), 829-834).

For the purpose of a cardiac fitness measurement, the system may maximize the SNR related to aortic valve closure by deeper sampling of larger vessels such as arteries and arterioles that maintain a stronger signal of aortic valve closure.

### Tissue-Sensor Interface

A prominent noise source for all sensing technologies is movement of the measurement device relative to the tissue. Device design can mitigate this issue, by protruding sensing components relative to the surface of the device such that they maintain consistent contact with the tissue.

For optical systems, device design can also reduce noise caused by ambient or stray light. Preferably, only light rays that have interacted with the tissue will be captured by the detector. However, light rays that have merely bounced off the skin or other surfaces, or that originate from environmental sources might also be detected and constitute a source of noise. Embodiments of the invention can include light-management components that control or restrict detected light. These components include but are not limited to physical blockers placed around the detector to limit the angles of light rays that can reach the photosensitive surface, optical elements (such as optical fibers or lenses) placed in front of the photodetector that similarly restrict the numerical aperture of the detector, and polarizers placed between the light source and detector at orthogonal orientations to limit detection of light rays that have only undergone surface reflections.

Additionally, ambient light cancellation (ALC) can be incorporated to remove interference from ambient light. ALC approaches detect light both when LEDs are active and inactive, allowing for compensation of signals in LED active periods by LED inactive periods. An example of ALC circuitry is disclosed by Kim et al (Kim, Jongpal, et al. “Ambient light cancellation in photoplethysmogram application using alternating sampling and charge redistribution technique.” 2015 37th Annual International Conference of the IEEE Engineering in Medicine and Biology Society (EMBC). IEEE, 2015).

### Size of Physiological Signal

Beyond changes to the operational parameters and configuration of the optical sensor system, the SNR can be increased by changing the size of the pulsatile signal.

The size of arterial pulsations can be increased by decreasing the vascular transmural pressure (TMP), that is, the pressure gradient across artery walls. At least three mechanisms are responsible for this enhancement in pulse size with TMP decrease: (1) decreases in TMP trigger arterial dilations through the local venoarterial reflex (VAR), (2) decreases in TMP trigger the myogenic response, i.e., the relaxation of the smooth muscles in artery walls, and (3) because vessel compliance is a function of TMP, decreases in TMP increase arterial compliance such that a given change in arterial pressure results in a large change in arterial volume. TMP can be reduced by applying external pressure at the measurement site or raising the elevation of the measurement site relative to the heart to decrease hydrostatic pressure.

Optimal external pressure is typically greater than the venous pressure but less than the arterial diastolic pressure; pressures beyond this point will begin to occlude flow and distort the pulse waveform. Based on the work of Balijepalli et al (2014), 95% of individuals aged 18-99 years have a diastolic pressure above 60 mmHg. If the sampling site is near or below the level of the heart, external pressures in the range of 50 mmHg can be appropriate to increase the magnitude of arterial pulsation;

The effect of TMP on pulse size is graded, thus any appreciable external pressure (e.g., greater than 5 mmHg) will produce some increase in the pulse. (Balijepalli, C., Lösch, C., Bramlage, P., Erbel, R., Humphries, K. H., Jöckel, K.-H., & Moebus, S. (2014). Percentile distribution of blood pressure readings in 35683 men and women aged 18 to 99 years. Journal of Human Hypertension, 28(3), 193-200).

By way of comparison, Brophy-Williams et al (2014) report that “sports compression” tights exert an interface pressure in the range of 5 to 30 mmHg, depending on the region of the lower limb and body posture. Coltman et al (2015) find that standard bra straps can exert ˜40 mmHg of pressure in static positions, and as much as 75 mmHg during high intensity activities. Thus, an external pressure of ˜50 mmHg is not outside the range of pressures exerted by standard garments, though is likely beyond the range of pressures produced by garments or devices intended to be intrinsically comfortable. (Brophy-Williams, N., Driller, M. W., Shing, C. M., Fell, J. W., & Halson, S. L. (2015). Confounding compression: The effects of posture, sizing and garment type on measured interface pressure in sports compression clothing. Journal of Sports Sciences, 33(13), 1403-1410). (Coltman, C. E., McGhee, D. E., & Steele, J. R. (2015). Bra strap orientations and designs to minimise bra strap discomfort and pressure during sport and exercise in women with large breasts. Sports Medicine—Open, 1(1), 21).

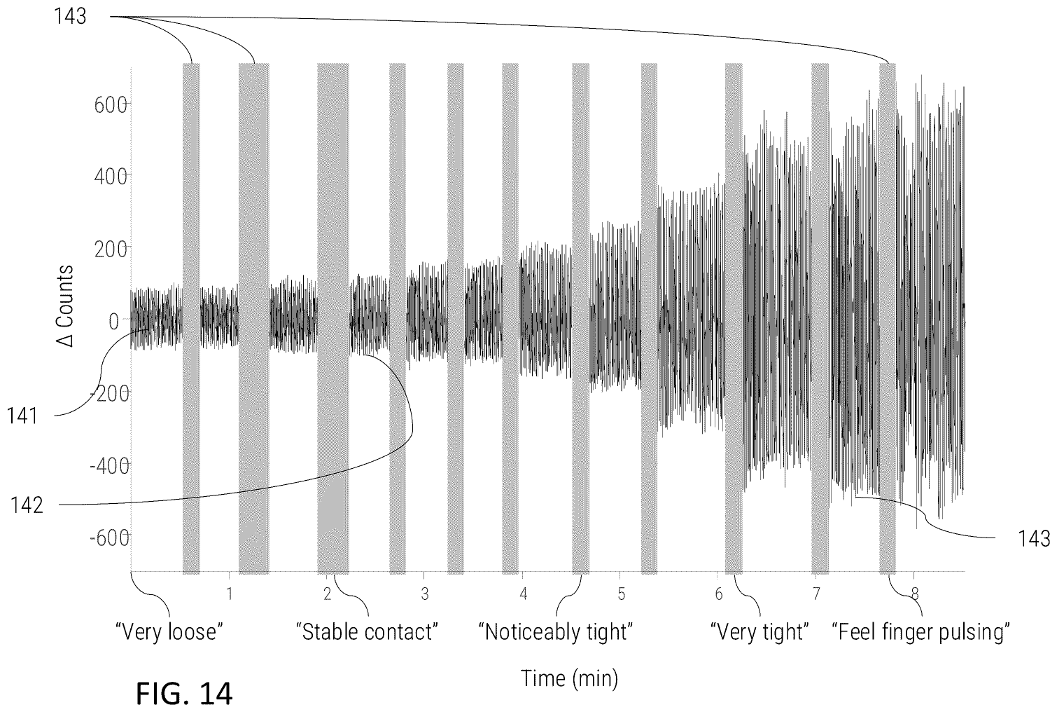

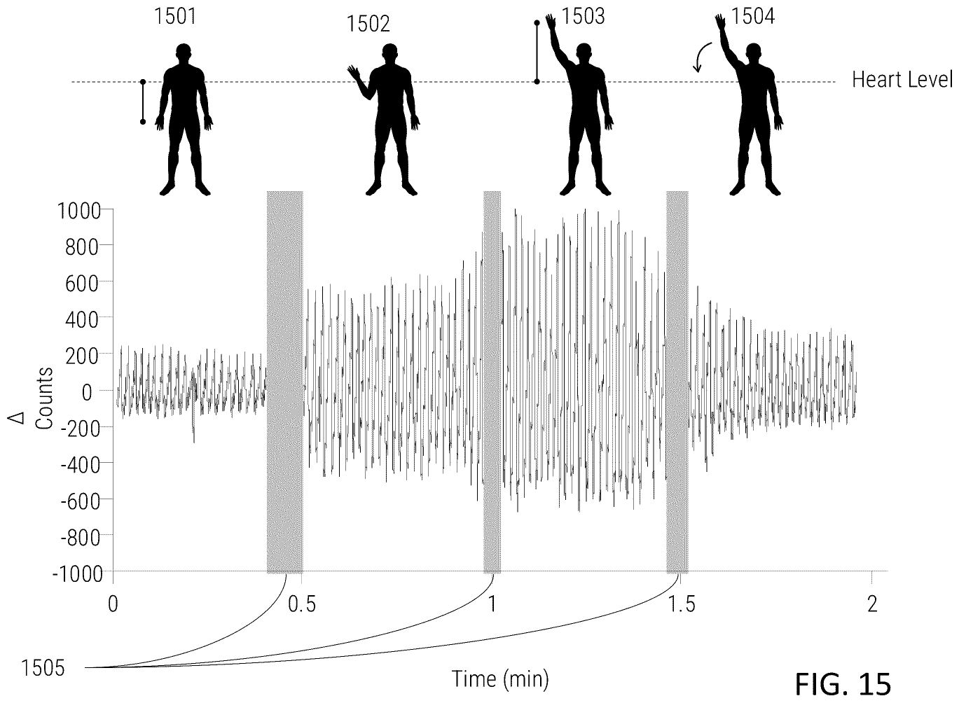

Examples of the effect of TMP on pulse size are shown in FIG. 14 and FIG. 15. FIG. 14 shows the detector signal from an adjustable PPG ring worn at the base of the finger. The measured signal has been band-pass filtered to focus on the pulsatile component. Roughly every 45 s, the ring is tightened incrementally on the wearer's finger via a ratcheting mechanism on the ring band. These tightening events are denoted by gray rectangles 103. The wearer's reported subjective experience associated with the different levels of tightness are indicated below the graph. Initially in period 101, the ring is reported by the user to be “very loose” and the magnitude of the pulse is ˜100 detector counts. After several tightening events the user reports that the ring makes “stable contact” with the finger. The pulse size at this period (102) is ˜150 counts. After this point, each tightening event increasingly changes the TMP through applied external pressure, as evidenced by the increase in pulsatile signal size. When the ring is reported by the user to be “very tight”, the pulse size increases to ˜1000 counts (period 103). After further tightening, the user reports feeling pulsations in the finger, an indication that the external pressure is approaching arterial diastolic pressure. Cumulatively, the tightening events produced a 10% reduction in the circumference of the ring and created a 10-fold increase in signal size is due to the decrease in arterial TMP caused by the increased external pressure at the sampling site.

FIG. 15 shows a second example of the effect of TMP on pulse size, in this case using manipulations in hydrostatic pressure to alter the TMP. FIG. 15 shows a band-pass filtered detector signal from a PPG ring worn at the base of the finger. The ring size is constant throughout the experiment, but the subject undergoes changes in arm positions, indicated by gray rectangles 1105. In period 1101, the arm hangs in a relaxed position at the subject's side. The sampling site is estimated to be 50 cm below the right atrium of heart, resulting in ˜37 mmHg of additive pressure distending the walls of the veins and arteries, due to the hydrostatic pressure exerted by the vertical columns of blood in these vessels. The pulse size in this period is just under 400 counts. In period 1102, the subject raises their hand such that the sampling site is roughly level with the shoulder. The change in vertical displacement with respect to the heart decreases the hydrostatic pressure, decreasing the TMP accordingly. The pulse size therefore increases by more than a factor of 2 to nearly 1000 counts. In period 1103 the subject extends their arm to a comfortable position above their head. The sampling site is now an estimated 67 cm above the right atrium, resulting in a hydrostatic pressure of roughly −50 mmHg. This reduces the TMP, which causes a further increase in the pulse size to roughly 1500 counts. In period 1104, the subject slowly lowers their arm down. As would be expected, the pulse size gradually decreases. FIG. 15 shows the patient in a standing position, but the same influences of TMP on pulse size are present with the rotation of the arm from the supine position.

Decreasing TMP at the sampling site provides the additional benefit of reducing physiological signals that are unrelated to aortic valve closure. A large source of physiological noise is venous blood. Since the venous system operates at relatively low pressures, it is quite susceptible to the local effects of volume perturbation during motion. The venous blood in the vascular bed will be easily deformed during subtle motion, changing light absorption and producing a significant source of in-band noise. This noise source can be managed by reducing the venous TMP to below zero, effectively collapsing the veins such that their volume is minimized. This not only stabilizes the venous contribution to vascular volume, but also reduces the overall absorbance of light by non-pulsatile sources.

The magnitude of the pulse signal can also be enhanced by increasing the cross-sectional area of the arteries and arterioles at the sampling site via vasodilation. This can be achieved by warming the tissue at the sampling site, for example, with a heating element embedded in the apparatus.

### Key Operational Elements and Systems

### Calculation of Interbeat Time Interval and Ejection Time Parameters

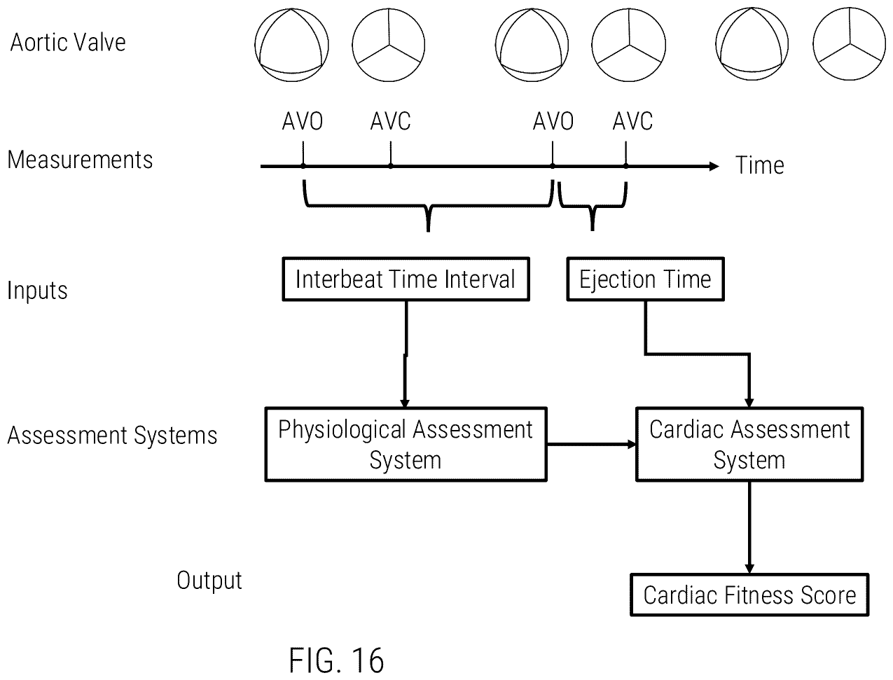

FIG. 16 shows the time course of aortic valve opening and closing, from which the interbeat time interval (IBI; the inverse of heart rate) and ejection time (ET) are determined. The measurement of these parameters is based on successive cardiac cycles. A cardiac cycle is defined as the performance of the human heart from the beginning of one heartbeat to the beginning of the next and, by requirement, includes an aortic valve opening and closing. It consists of two periods: one during which the heart muscle relaxes and refills with blood, called diastole, following a period of robust contraction and pumping of blood, called systole. For interbeat time internal determination, the measure is between the aortic opening and the next aortic opening in successive cardiac cycles. These calculated parameters can be used by the assessment systems for the determination of basal physiological status and cardiac fitness.

### Analysis Methods

The physiological and cardiac fitness assessment systems utilize sophisticated analysis methods to determine the physiologic state and cardiac fitness. The assessment system conducts a series of mathematical data processing steps without patient/user involvement. The analysis method can include many classes of models but can be broadly broken into “prediction models” and “matching models.” Prediction models are constructed by determining the relationship between data or data features and desired output; once the relationship is determined, the model can be applied to novel data with no reliance on training or reference data. These models are distinct from matching models, which rely on a pre-existing library of training or reference data. A matching model determines the proximity of novel data to reference data to produce the desired output. Examples of prediction models include regression models, where features are mapped to outputs through linear or non-linear relationships, as well as some machine learning models, in which more complex data representations are mapped to the desired output. In these approaches, often referred to as “deep learning models”, the useful features and representations are essentially learned by the model in training, along with the function that maps the inputs to the desired outputs. Because the relationship between input and outputs is often quite complex (involving thousands of weights in multiple hierarchical layers), the engineer or architect of the model might be completely unaware of the features or information that the model has extracted or how and why that information is combined to form the output. A hierarchical model is a model in which lower levels are sorted under a hierarchy of successively higher-level units. For all kinds of models, the desired output can be a continuous variable (e.g., a cardiac fitness score) or a binary variable that takes on values of zero/one, indicating the presence or absence of a specific condition or state (e.g., a basal physiological state). In some embodiments of this invention, the assessment systems can use as inputs features or calculated parameters extracted from measured signals that contain the relationship between the status of the aortic valve and some measurement of time. In other embodiments, the assessment system can use as input measured signals from the sensors, in raw or conditioned data representations that contain the relationship between the status of the aortic valve, pulse morphology information, and a measure of time, referred to as systolic interval information. These different types of analysis methods can be used independently or in combination.

### Physiological Assessment System

The physiological assessment system is broadly defined as the hardware and software that performs the calculations, logical operations, and analysis methods to determine if the patient's physiological state is appropriate for determining an accurate basal cardiac fitness. The assessment may require calculations and comparisons with prior measurements. The operation of the physiological assessment system does not require user engagement. Obtaining cardiac vagal control necessitates a low sympathetic tone, and no operational responsibilities should be placed on the patient. For many individuals, managing a complex or unfamiliar process would result in stress causing an increase in sympathetic tone, a condition inconsistent with ideal measurement conditions. The physiological assessment system manages the complex calculations of the system, processes the incoming data streams, and completes the mathematical operations needed to perform the analysis methods used to determine the patient's physiological status. The physiological assessment system may use a variety of analysis methods.

### Cardiac Fitness Analysis System

The cardiac fitness determination system determines cardiac fitness by conducting an analysis process of cardiometric information. The cardiac fitness determination system is broadly defined as the hardware and software that analyzes the cardiometric signals. It includes any process that takes defined inputs and applies calculations, analysis methods, or a designated set of steps to determine the desired output. A cardiac fitness assessment system can also include additional inputs, such as body position, breathing rate, time of day, ancillary information, or other information about the user or the test environment. The cardiac fitness assessment system can use a multitude of analysis methods based on different features or algorithms and combine the results to provide singular or multiple outputs. The output of the determination analysis system is an assessment of cardiac fitness that can be used by either the patient, provider, or coach

### EXAMPLE EMBODIMENTS

### Locations of Assessment

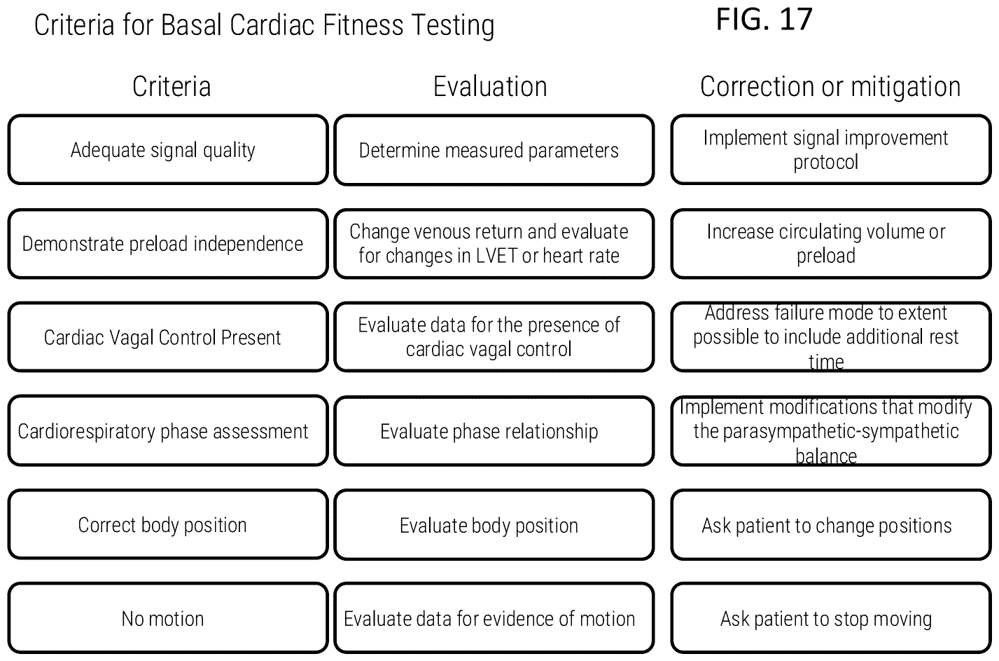

Multiple locations for data acquisition exist, but three general locations, scenarios, and their method of operation and associated hardware will be described. The first scenario is the determination of basal cardiac fitness in the medical clinic or associated facilities. The second scenario is the determination of basal cardiac fitness in the home over a limited duration, a couple of days. The third scenario is a variant of the second but is specifically associated with the continuous use of the system and the procurement of data in an observational manner over longer periods. FIG. 17 is a general list of some criteria that can be used to ensure the quality of the measurement. The figure shows the general criteria, a proposed evaluation approach, and potential mitigation activities.

### Use of Multiple Criteria

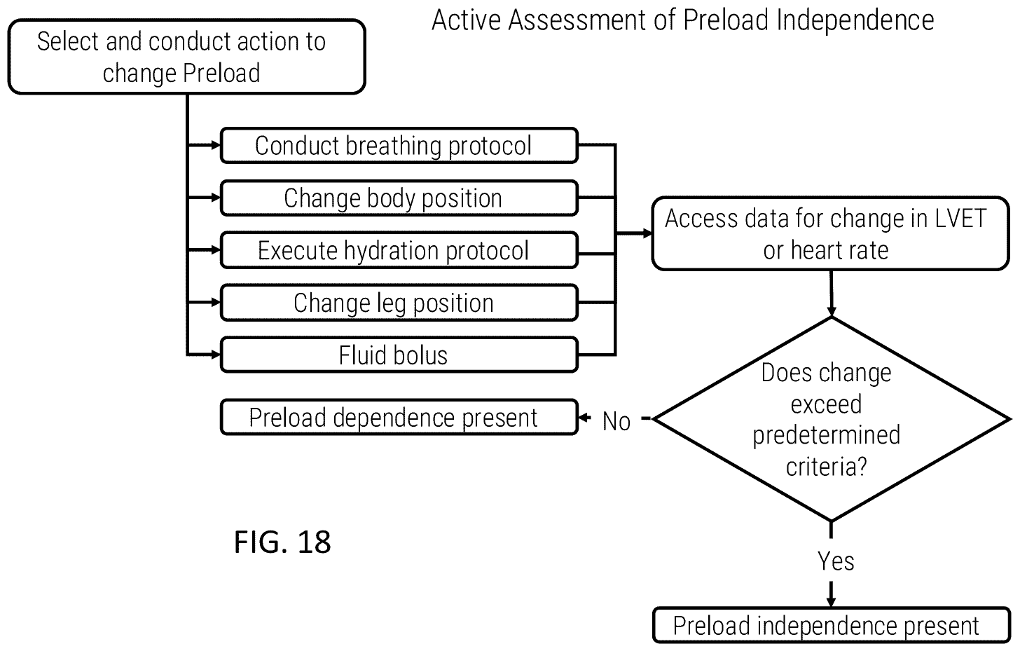

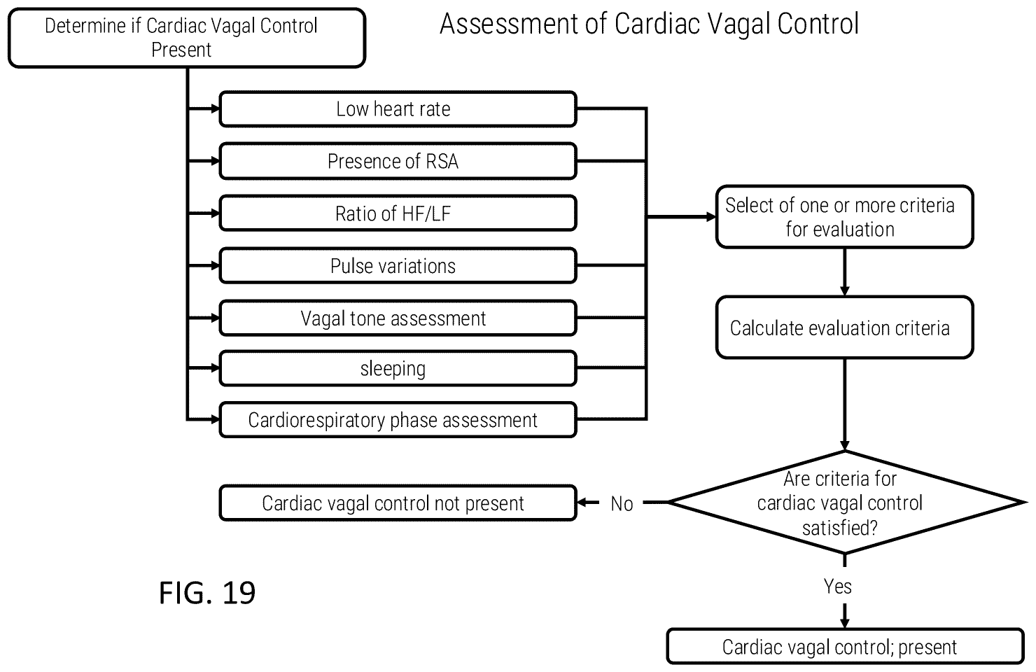

The accurate determination of basal cardiac fitness is a measurement activity that requires a moderate degree of process control or quality control. The location of measurement, clinic or home influences the criteria that can be reasonably implemented. The inadequate management of key activities could result in an inaccurate assessment. FIG. 17 is a list of some general criteria that could be used. These general criteria can be refined further. For example, FIG. 18 is an example of criteria and a general flow chat that could be used for the active assessment of preload independence. FIG. 19 is an example of potential criteria to use for the determination of cardiac vagal control. The examples are included to illustrate the array of criteria that can be used as well as their importance.

### Medical Clinic Testing

### Example Method and Considerations for Medical Clinic Testing

The assessment of basal cardiac fitness in the clinic allows the interaction of the patient with a medical professional and the ability to implement testing, mitigation, and correction activities. Specifically, the ability to get a definitive assessment of preload independence by altering venous return through an action of the patient or the medical professional enables the assessment of the criteria associated with preload independence. However, testing in the clinic has some difficulties as many patients suffer from “white coat syndrome” and often have an increased sympathetic tone in response to the medical clinic. Therefore, attention must be given to ensuring that the patient is comfortable, resting, and in an unstressed state so a basal physiological state is obtained before capturing cardiometric parameters. The medical clinic may enable the use of multiple measurement systems, the use of a larger battery, and sensors that would not be easily worn while sleeping at home. The system may have an input and display station that helps coordinate the measurement process. The key consideration associated with clinic testing is the desire to obtain the measurement in a timely fashion.

The measurement sequence begins with obtaining relevant patient information and the attachment of the noninvasive sensor system to the patient in a comfortable manner. The patient is positioned in a supine position and instructed not to move during the testing period. Following the attachment, the objective is to satisfy a defined set of criteria associated with the physiological state, signal quality, and patient participation. FIG. 17 is an illustration of potential criteria, valuation methods, and correction or mitigation activities. The criteria listed are potential examples of criteria that could be used either singularly or in combination to ensure that conditions of preload independence, cardiac vagal control, and adequate signal quality have been satisfied. For the purpose of this illustration, assume all criteria are used for determining a basal physiological state.

Following the attachment of the system, the overall signal quality can be assessed, and the parameters of measurement modified to facilitate improved signal measurement by the sensor system. Specifically, the vascular transmural pressure can be decreased to improve pulse size. The process can involve changing hydrostatic pressure or generating more external pressure at the measurement site. Due to time limitations in the clinic, the ability to improve signal strength over the testing period is a valuable capability.

Preload independence can be accessed first as it is not dependent on a restful state like cardiac vagal tone. Preload independence can be accessed via changes in venous return. FIG. 18 is a flow chart illustrating possible methods for determining preload independence in the clinic. A passive leg raise is a commonly used method for changing venous return. Thus, the medical practitioner would implement a passive leg raise, and the physiological assessment system would determine if preload independence is present. Changes in LVET or heart rate can be used to determine the presence of preload independence.

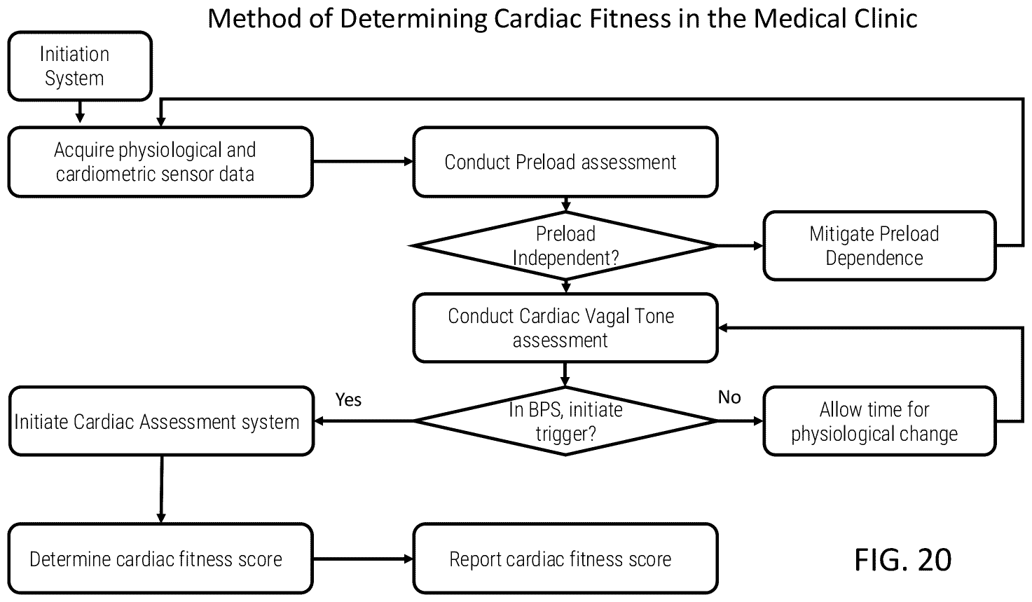

After the determination of preload independence, interactions with medical personnel can be stopped, and the patient can be allowed to rest while the sensor system acquires physiological signals and the physiological assessment system monitors for the presence of cardiac vagal control. The system can access a variety of physiological measurements for the assessment of cardiac vagal tone to include: sinus arrhythmia, heart rate variability, vagal tone, the balance between the sympathetic and parasympathetic systems, resting state, low heart rate, and pulse variations. FIG. 19 is a flow chart illustrating potential methods for satisfying cardiac vagal control. The method, as illustrated, is based on the concepts communicated in FIG. 11. In practice, the system may operate in a continuous sampling mode until cardiac vagal control is present. This process may necessitate the patient to relax in the exam room over time until the criteria are satisfied. FIG. 20 is a flow chart of the steps in the testing method. The flowchart provides for mitigations if the patient is not in a basal physiological state.

### Example Apparatus for Clinic Testing System

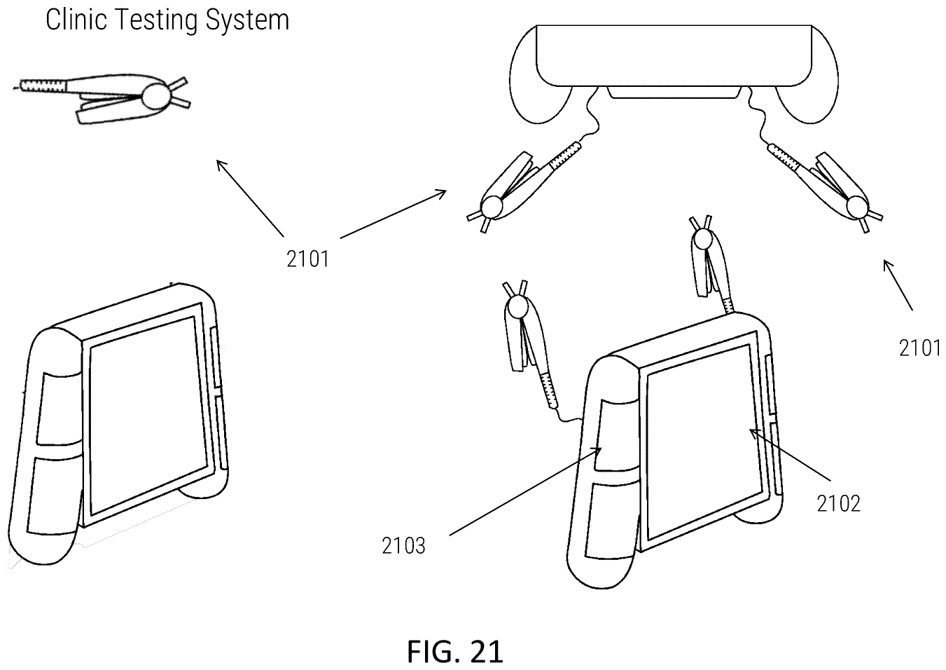

FIG. 21 is an example embodiment of a potential cardiac fitness test system. The measurement system is designed so the patient can be comfortable in the supine position with arms relaxed on the chest or arms beside the patient (patient not shown). The system can contain two optical measurement systems in the form of finger clips that enable the procurement of physiological and cardiometric signals sensitive to blood volume, flow, or pressure in the fingertip of the patient (2101). Two sensors system are illustrated to help procure high-quality measurement signals, although only one sensor could be used. The finger clip sensors can be equipped with mechanisms that decrease the transmural pressure by compressing the finger (not shown). Although not required, the placement of the hands on the chest creates a slight hydrostatic pressure difference that would decrease transmural pressure but may feel awkward to some patients. The illustration shows the finger clip devices attached to a user input screen/device and an unattached version. In the unattached finger clip version, the finger clips are wirelessly connected to the user input screen/device. In either scenario, the system contains a screen (2102) for user input, the display of measurement progress, and for providing results. Additional measurements can be added to the system as needed. For example, a contact pad for ECG measurements is illustrated (2103).

### Example Flow of Information in Medical Clinic Testing

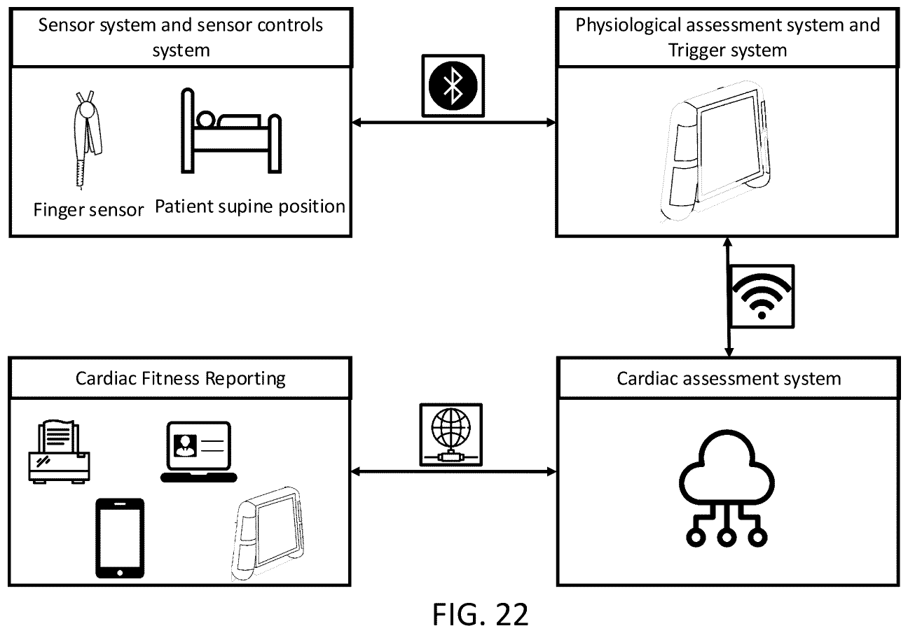

Multiple avenues exist for information flow, with FIG. 22 as an example illustration. The sensor and sensor control system is in the finger clip sensors. The finger clips are Bluetooth connected to the display/input device that contains the physiological assessment system and the trigger system. Upon determining the presence of a basal physiological state, the sensor data and a trigger signal are communicated via Wi-Fi to the cloud computing capability where the cardiac assessment system is located. The cardiac assessment system determines the cardiac fitness score, and the cardiac reporting system communicates with a mobile application, the electronic medical record, and a printer for a generation of a physical copy. During clinical use, the finger sensors will get dropped and damaged so the amount of processing hardware in these devices should be minimized. Thus, the physiological assessment system processing hardware and software is in the display/input device, which in turn may be in a docking station. The cardiac assessment system is in the cloud computing location as the requirements for determining aortic closure, conducting historical comparisons, and completing the cardiac fitness report require more sophisticated hardware, and software and access to other information sources.

### Limited Duration Home Testing

### Example Method and Considerations for Limited Duration Home Testing

The assessment of basal cardiac fitness in the home provides the opportunity to make measurements while the patient is sleeping and obtain multiple measurements over a longer duration than is reasonably available in the clinic setting. The home setting affords the ability to determine preload independence by observational activities. In a proposed embodiment, the system can identify those periods of low heart rate and respiratory sinus arrhythmia as potential data acquisition periods. Upon identification of such a physiological state, the system can trigger the modification of operational parameters for improved signal-to-noise such that aortic opening and closing information is obtained or may engage other sensors in signal acquisition. The resulting measured cardiometric signals can then be processed by the cardiac fitness assessment system to determine a basal cardiac fitness score. The resulting measurements can be compared with other measurements obtained during a single night of sleep, over several nights, or over a more extended period. The criteria for initiation of a trigger event are based on determining the presence of a basal physiological state. Potential information to facilitate the procurement of measurement signals during a basal physiological state can include lack of movement, sleep stage, and cardiorespiratory phase relationship. If the criteria are not satisfied, several mitigations are provided, including acquiring data at a different time.

In an example use scenario, a patient is instructed by their medical provider to use the measurement system while sleeping for a week. The medical provider instructs the patient to perform a hydration protocol (e.g., drinking 500 ml of fluid before bed) to help create a physiological state that satisfies the constraint of preload independence. The patient attaches the system for continuous or semi-continuous measurements while sleeping. The attachment of the system to the patient could serve as the initiation event.

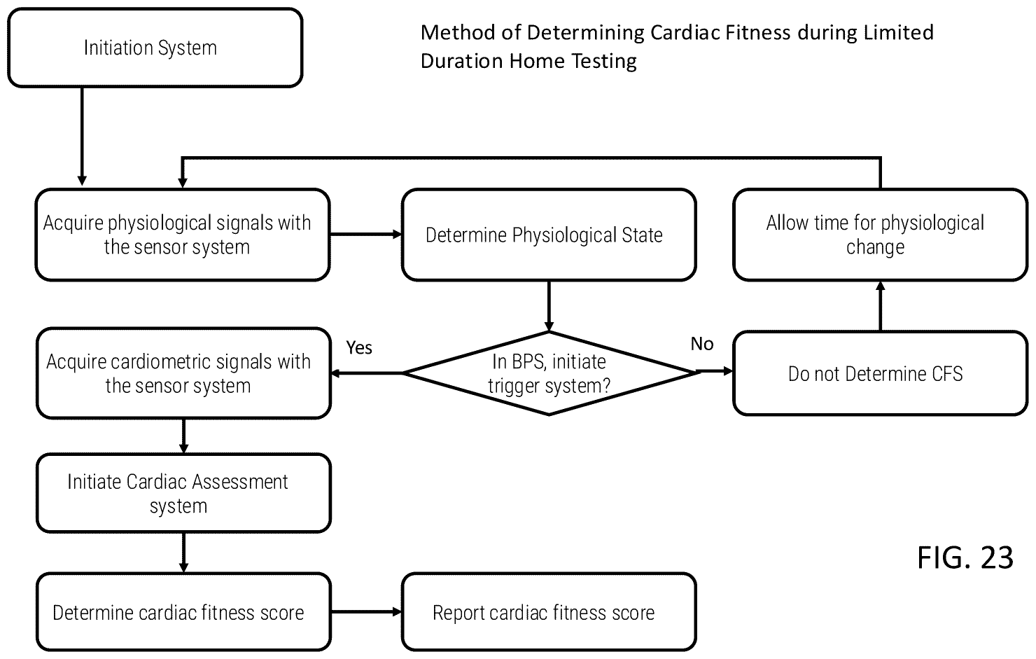

FIG. 23 illustrates a potential measurement sequence. The patient would initiate the system prior to bedtime, and the system would begin to obtain physiological signals. The resulting physiological signals would be processed from the presence of a basal physiological state. The system may use pre-determined thresholds for interbeat time intervals and interbeat variability if no prior physiological data on the patient is available. Upon detecting a basal physiological state, the trigger system is initiated, and the sensor control system changes operational parameters for the acquisition of cardiometric signals. After obtaining cardiometric signals over a reasonable time period, the sensor system could pause and re-initiate physiological signal acquisition after a defined period.

For example, the system could be in pause mode for the first 30 minutes following the initiation event as the patient falls asleep. Then assuming that a basal physiological state is present, the system could measure cardiometric signals for 3 minutes. The system could then pause for an hour and reinitiate physiological signal measurement. The system could be programmed to obtain a maximum of 6 cardiometric signal blocks each night. The use of an intermittent signal acquisition could help limit battery drain and the total amount of memory on the system.

### Example Apparatus for Limited Duration Home Testing

Multiple possible embodiments exist that could be used for home testing. In general, any sensor system that does not interfere with sleep and will not be easily dislodged during sleep is applicable. All the PPG/SPG measurement configurations illustrated in FIG. 12 could be used. For illustrative purposes, a muti-measurement technology sensor system with defined operating modes will be described. The sensor system as illustrated in FIG. 23 and combines the sensing technologies of electrocardiogramanocardiography, and speckle plethysmography. The system uses a chest strap to locate the system in close proximity to the heart (2401). The system has contact pads for sensing the EKG, 3403, a microphone for phonocardiography measurements (2405). The speckle plethysmography system uses a multi-emitter and multi-detector configuration for the procurement of the best pulse waveform. As shown, the system utilized emitters (2402) and an array of detectors (2404).

In operation, the EKG signal is the source of physiological measurement signals for the determination of a basal physiological condition. Upon detection of a basal physiological state, the trigger system initiates the measurement of cardiometric signals. As configured, the EKG signal combined with the phonographic signal enables the calculation of PEP. The speckle signal enables the measurement of aortic opening and closing and the calculation of ejection time. Thus, the system measured two cardiometric signals and enable the the transfer of measured systolic time interval information or the transfer of systolic time interval parameters. The cardiometric signal measurement period could be for one minute. After signal acquisition, the sensor system may pause signal acquisition for an hour with a subsequent return to physiological signal measurement and the detection of a basal physiological state

### Example Flow of Information in Limited Duration Home Testing

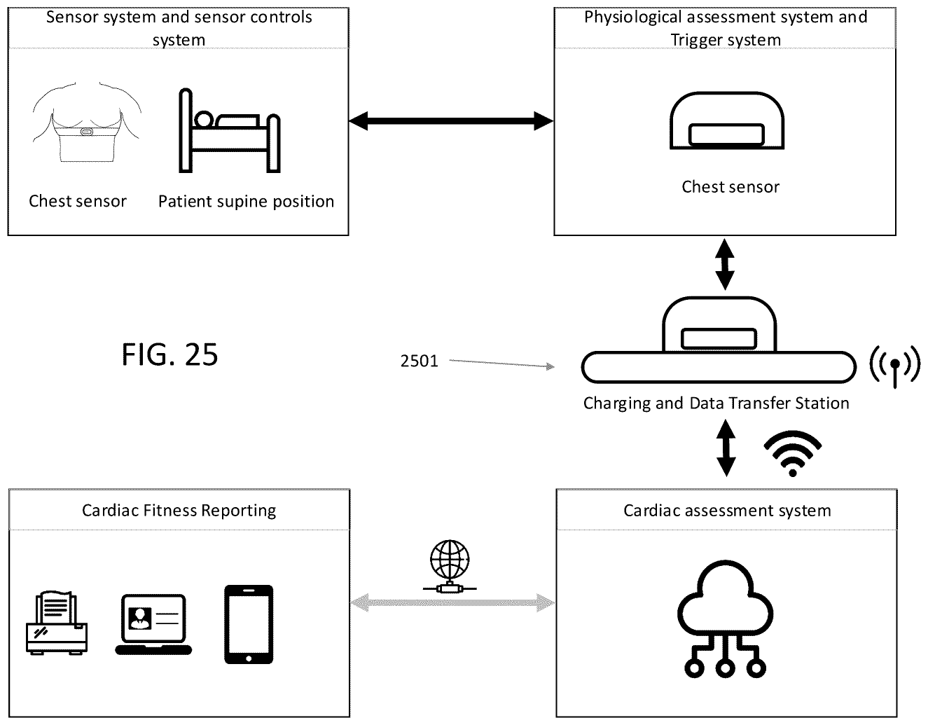

Multiple avenues exist for information flow, but FIG. 25 is an example illustration. The chest strap unit contains the sensor system, the sensor control system, the hardware and software for physiological assessment, and the trigger system. The signal measurement sequence is controlled by the microcontroller located in the chest strap unit. After a night of signal measurement, the chest strap unit is placed on a charging and data transfer device (2501), and the acquired data is transferred to the cloud. The daily transfer of data and recharging of the device limits the need for multi-day memory and power. After completion of the assessment period, assumed to be about a week, the sensor system can be mailed or physically returned. The cardiometric data obtained during periods of basal physiological status can be processed by cloud computing hardware and software. The examination of multiple measurements during each night and the aggregation of multiple days of measurement creates a repository of cardiometric data upon which to generate a cardiometric fitness report based on robust statistical methods. The use of robust statistical methods enables the use of statistical methods, models, or definitions that are not unduly affected by outliers or other departures from model assumptions. The resulting cardiac fitness score and associated report can be communicated to the patient, provider and stored in the patient's medical record.

### Example Method for Longer-Term Monitoring

### Example Method and Consideration of Continuous Home Monitoring

Accessing heart function over time has significant value in those patients at risk for developing heart failure. In addition to accessing heart function, such a wearable system could measure other cardiac relevant parameters such as activity level, sleep duration and quality, and oxygen saturation. The ability to add a medically relevant measurement of cardiac function to a more standard activity-tracking wearable has significant value for the patient. In developing such a system, the size and expense of the device become key considerations. Processing power should be minimized for expense, power, and size consideration. Power considerations are also important as batteries impact both size and weight. Thus, the proposed method of sampling seeks to create a method that enables the realization of a small, inexpensive, and long battery life system.

In practice, many wearable devices, especially ring systems, take data systematically during the day and night. For cardiac assessment, the proposed method will focus on periods most likely consistent with basal physiological status. Such periods are associated with a supine position and sleeping. Thus, an objective of the system is to identify periods likely to represent a basal physiological state and obtain cardiometric data in an efficient manner. In practice, the system used a logical process to identify those periods when the patient is most likely to be in a basal physiological state. During such a period, the system will obtain physiological data and conduct a preliminary physiological assessment. If criteria suggesting a basal physiological state are present, then the sensor system is triggered to obtain cardiometric data. The measured physiological and cardiometric signal data is subsequently transferred for additional processing. Additional processing can include further evaluation of the physiological data to ensure that a basal physiological state was present. If a basal physiological state is confirmed, then the trigger system initiates the processing of the cardiometric data for the determination of a cardiac fitness score.

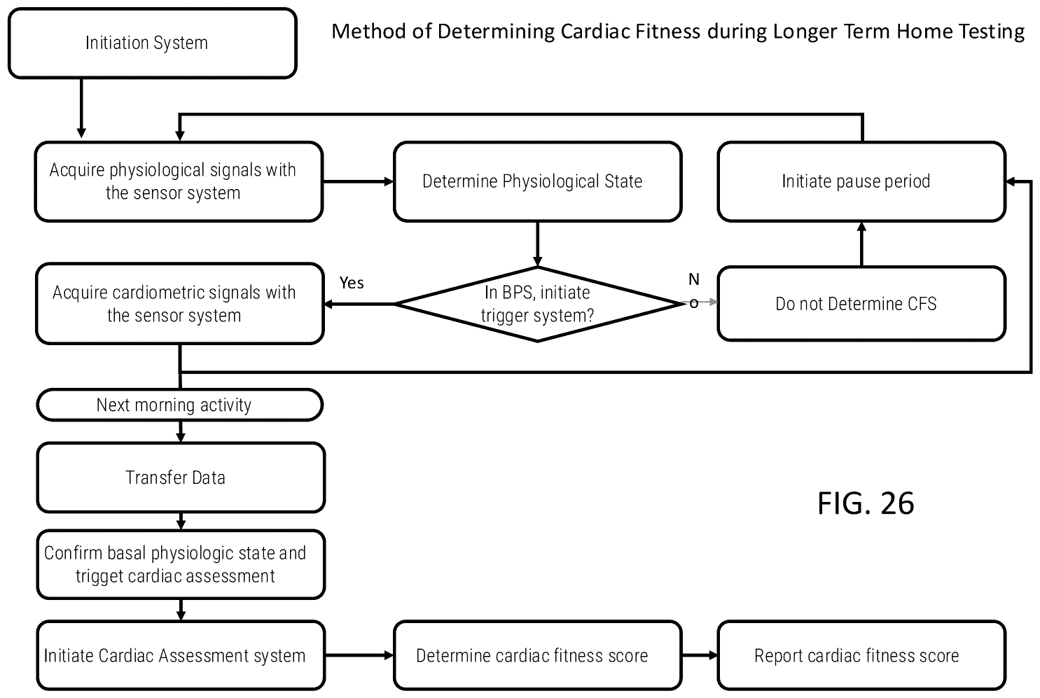

For the purpose of this illustrative embodiment, the wearable system will be a ring and the overall measurement method is illustrated in FIG. 26. A ring system places significant constraints on size and power consumption. Thus, an objective is to focus data acquisition during periods of deep sleep as it is most consistent with a basal physiological state. Humans spend the most time in deep sleep during the first half of the night. During the early sleep cycles, deep sleep stages commonly last for 20-40 minutes. As sleep continues, these stages get shorter, and more time is spent in rapid eye movement (REM) sleep, which is undesirable due to increased sympathetic tone. As the wearable system tracks general activity, the sensation of movement and a supine body position could be used to assume sleep has been initiated. After the cessation of movement, the system waits 30 minutes and acquires physiological data. The physiological assessment system located on the ring does a preliminary assessment based on interbeat time intervals. If the interbeat time interval exceeds a fixed threshold, then the trigger system initiates cardiometric data acquisition of a short period, assuming 10 to 20 cardiac cycles. The cardiometric signal measurement mode is defined by different operational parameters that are communicated from the sensor control system to the sensor system. The operational parameters are likely to increase the sampling frequency of data acquisition and the intensity of the light emitters. After this measurement sequence has been completed, data acquisition by the sensor system is paused, assuming 50 minutes. After the pause, the measurement and assessment sequence is repeated. The system only operates during the first half of sleep with the objective of maximizing the probability of having the patient in a basal physiological state.

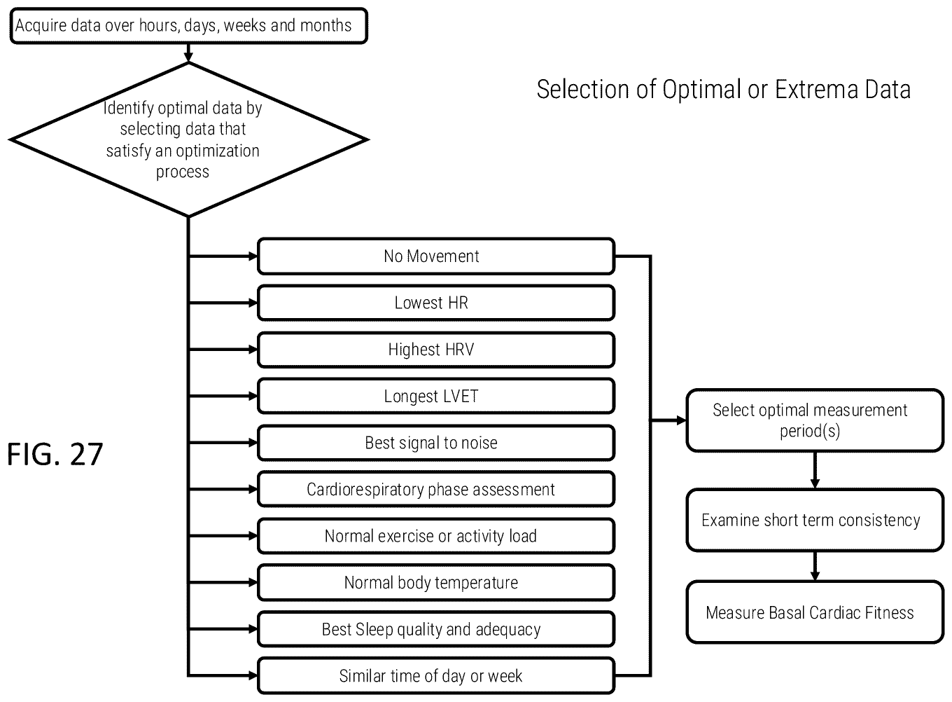

For this illustration, it is assumed that the cardiometric measurement signals are transferred for subsequent processing. The transfer of the total measurement signal enables both the calculation of systolic tie intervals as well as the processing of the data by deep learning tools. The operation of the system seeks to transfer only appropriate data, and an amount needed to determine cardiac fitness but not more data than might be desired. The transferred data can be processed for the identification of maximal ejection time. Changes in sympathetic tone, increased heart rate, and preload dependence shorten left ventricular ejection time. Thus, identifying the longest or a group of long left ventricular ejection times over a measurement or assessment period may be used to define basal cardiac fitness level. Determining these extrema conditions can be done on the cardiometric data measured via the signal measurement process defined above. Determining the ideal measurement conditions (extrema conditions) can include several parameters beyond ejection time. FIG. 27 illustrates several potential criteria that could be used singularly or in combination to identify physiological and cardiometric extrema conditions upon which a basal cardiac fitness test can be conducted.

### Example Apparatus for Longer Term Home Monitoring

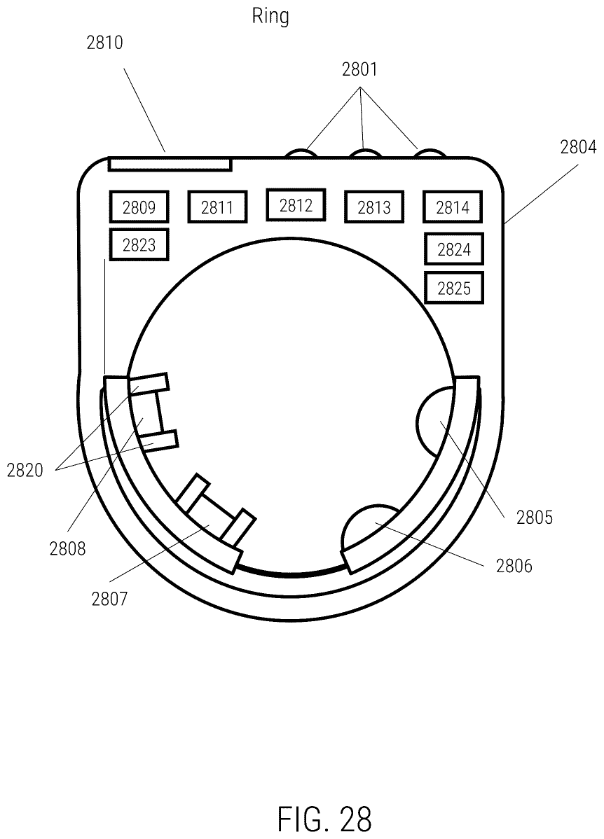

FIG. 28 shows an illustrative embodiment of an apparatus (2804) using optical measurement technologies capable of making a cardiac fitness measurement based on measuring signals containing information on aortic valve opening and closing. In the example embodiment, the apparatus is configured as a ring to be worn on a finger. The apparatus includes one or more of the operating systems described below. Each system's functional element(s) are described, though additional capabilities can also be present. The description of apparatus and operation is described for the systematic data acquisition mode.

The apparatus may enable a user to perform an initiation trigger (2810). The apparatus includes an optical measurement system comprising one or more emitters (2805 and 2806) and one or more detectors (2807 and 2808). The optical system emits photons into the tissue at a sampling location and detects photons that have interacted with the tissue. In this embodiment, physical blockers (2820) surround the detectors to limit the influence of stray light. The emitters can have the same emitting wavelength or different wavelengths. A given emitter can also represent a package of LEDs, capable of emitting a plurality of wavelengths. The detectors can be the same or different regarding their active area, spectral sensitivity, or other parameters. The optical sensor system can be configured to perform time-division multiplexing and de-multiplexing, such that signals from a plurality of wavelengths can be acquired during the same acquisition period. The optical sensor system can be further configured to perform ambient light cancellation.

The apparatus includes a sensor control (2823) for the management of operational parameters of the optical measurement system. Operational parameters include parameters of the optical system that can be configured to include an emitter and detector selection, wavelength selection, sampling frequency, detector integration time, ambient light cancellation, and sampling duration. For example, during a cardiac fitness measurement, when detection of aortic valve opening and closing is required, the following operational parameters for the optical sensor system shown in FIG. 28 can be changed to enable the use of both emitters (2805 and 2806) with a near-infrared wavelength (e.g., peak wavelength of 940 nm) at near maximal intensity (e.g., drive current of 60 mA); use of detector 2008 to encourage a long optical path with a deep sampling of arteries and arterioles, with a maximal integration time of 100 μs; sampling frequency of greater than 100 Hz; acquisition duration of 30 seconds. The above operational parameters are provided for illustration only; variations of these parameters can also be suitable. When a cardiac fitness measurement is not taking place, the data acquisition system can specify different operational parameters. For example, if the heart rate is determined, the operational parameters can be altered to reduce power requirements and conserve battery life. Such operational parameters can include the use of a single emitter 2006 emitting green light (e.g., peak wavelength of 530 nm) at sub-maximal intensity (e.g., 15 mA); use of detector 2007 to encourage a short optical path where photons largely interact with the capillary bed; sampling frequency of 16 Hz. During periods where no optical measurements are required, the data acquisition system can also fully inactivate the optical sensor system (achieving an effective sampling rate of 0 Hz for all detectors and drive current of 0 mA for all emitters) to further conserve power.

The ring sensor can further comprise a signal suitability system (2812), which determines a metric indicative of the suitability of the acquired measurement signals for cardiac fitness determination such that a reliable result will be generated. The suitability determination can be based on various factors, including the stability and consistency of the raw or processed measurement signals, the magnitude of motion as determined with the motion sensor system, and the estimated degree of motion contamination in the detector signals. The signal stability system can use outlier detection methods, anomaly detection methods, probability models, or other techniques to assess suitability. The signal suitability system can be configured to determine the cause of signal unsuitability and provide this diagnostic information to the user or patient via a feedback system such that corrective action might be taken. Additionally, the signal suitability system can be configured to provide information to the data acquisition system such that changes in operational parameters can be implemented to improve the quality of measured signals.

A motion sensor system, e.g., accelerometer (2809), obtains motion information at the sampling location. In alternative embodiments, the motion sensor system can comprise sensors that quantify motion in at least one dimension, such as accelerometers, gyroscopes, magnetometers, barometers, and altimeters. One or more of these sensors can be present in an inertial measurement unit (IMU). The sleep detection system (2825) can use parameters from the motion sensor system (2809) and the body position system (2813) to determine the presence of sleep. The body position assessment system can use physiological signals and accelerometer data to determine body position. The physiological assessment system (2811) determines the patient's physiological state by evaluating the physiological signals measured by the sensor system. In the illustrated embodiment, the trigger system (2814) obtains signals from the physiological assessment system to initiate a change in the operation of the sensor control system (2823).

In the described embodiment, the cardiometric measurement signals are transferred by a Bluetooth receiver (2824) to a smartphone or other transfer system for subsequent processing.

A final element of the ring may include a feedback system comprising display LEDs (2801) to provide feedback to the patient regarding the general operation of the device including battery life, completion of cardiac fitness measurement, etc.

### Example Flow of Information in Long-Term Home Monitoring

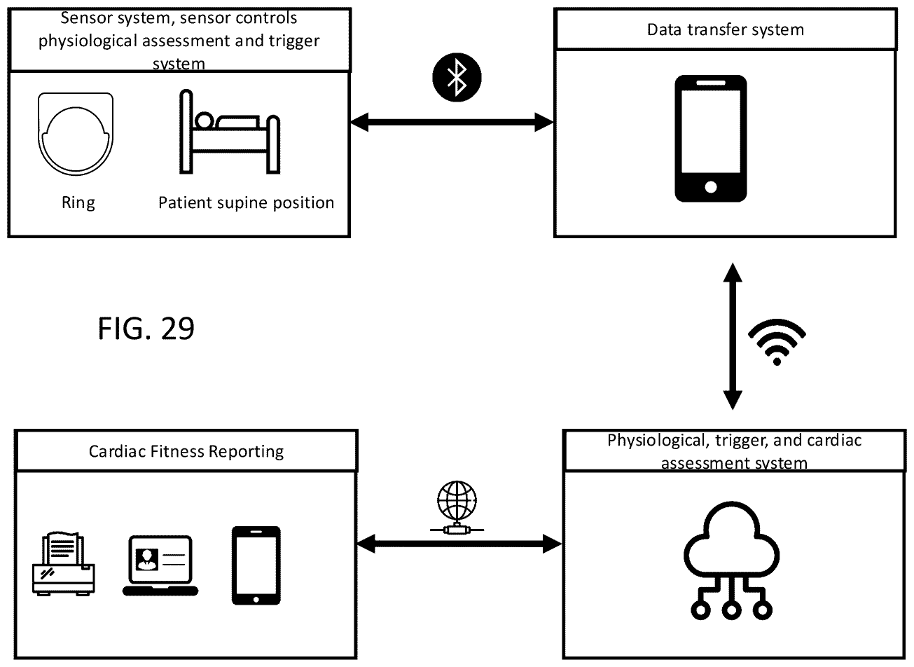

Multiple avenues exist for information flow, but FIG. 29 is an example illustration. The ring contains the sensor system, the sensor control system, the hardware and software for elements of the physiological assessment, and the trigger system. The signal measurement sequence is controlled by the sensor control system located in the ring. After a night of signal mesurement, the user can initiate synchronization activity with the ring to a Bluetooth-enabled smartphone. The phone correspondingly transfers the data to the cloud for completion of physiological assessment and determination of the cardiac fitness score. The calculated score can be transmitted to the patient's phone, the patient's medical record of the patient's provider. Alternatively, the cardiac fitness cores may be aggregated over an assessment period, approximately a month, and transferred to the patient and provider. The examination of multiple measurements during the assessment period and the aggregation of multiple weeks of measurement creates a repository of cardiometric data upon which to generate a cardiometric fitness report based on robust statistical methods.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those skilled in the art.

What is claimed is:

1. An apparatus for determining the cardiac fitness of a user, comprising:
(a) a noninvasive sensor system, comprising one or more cardiovascular sensors configured to produce a signal that indicates a time of opening and closing of the user's aortic valve;
(b) an initiation system, configured to detect an event indicating a cardiac fitness test is to be initiated;
(c) a sensor control system responsive to the initiation system configured to operate the noninvasive sensor system at a first set of operational parameters to produce a first measurement signal that indicates the times of opening and closing of the user's aortic valve during two or more successive cardiac cycles;
(d) a physiological assessment system configured to determine the presence of a basal physiological state from the first measurement signal based on (1) an interbeat time interval between successive openings of the user's aortic valve from each of two or more cardiac cycles, and (2) a variability between two or more interbeat time intervals,
(e) a trigger system, responsive to the physiological assessment system;
(f) a cardiac fitness assessment system responsive to the trigger system configured to activate when the trigger system indicates that a basal physiological state is detected and further configured to determine a cardiac fitness score from the first measurement signal based on an ejection time interval between an opening and an immediately subsequent closing of the user's aortic valve;
(g) a cardiac fitness reporting system configured to report the first cardiac fitness score.

2. The apparatus of claim 1, wherein the sensor control system is responsive to the trigger system and is configured to operate the sensor system at a second set of operational parameters to produce a second measurement signal when the trigger system indicates that a basal physiological state is detected, and wherein the cardiac fitness assessment system is configured to determine a cardiac fitness score from the second measurement signal.

3. The apparatus of claim 1, wherein the sensor system comprises optical emitters and detectors.

4. The apparatus of claim 1, wherein the noninvasive sensor system includes at least one of the following: electrocardiogram sensor, phonocardiogram sensor, seismocardiogram sensor, ballistocardiogram sensor, or echocardiogram sensor.

5. The apparatus of claim 1, wherein:
(a1) the noninvasive sensor system comprises an optical measurement system comprising (i) one or more optical emitters configured to emit light toward a measurement region of the user and (ii) one or more detectors configured such that light reaches the detectors from the one or more emitters after the light from the emitters has interacted with the measurement region;
(c1) the sensor control system is configured to operate the one or more emitters and the one or more detectors at a first set of operational parameters to detect changes in blood flow or blood volume to produce a first measurement signal that is indicative of opening and closing of the user's aortic valve;
(e1) the trigger system is configured to respond to the presence of a basal physiological state as determined by the physiological assessment system.

6. The apparatus of claim 5, wherein the sensor control system is responsive to the trigger system and operates the sensor system at a second set of operational parameters to produce a second measurement signal when the trigger system indicates that a basal physiological state is detected, and wherein the cardiac fitness assessment system is configured to determine a cardiac fitness score from the second measurement signal.

7. The apparatus of claim 5, wherein the optical measurement system includes a speckle plethysmography sensor.

8. The apparatus of claim 5, wherein the optical measurement system includes a photo plethysmography sensor.

9. A method for determining a basal cardiac fitness of a user in an unstressed state, comprising:
(a) providing a noninvasive sensor system configured to detect changes in blood volume or blood flow in a measurement region of the user, where the changes are indicative of opening and closing of the user's aortic valve;
(b) acquiring a measurement signal from the noninvasive sensor system;
(c) determining from the measurement signal an ejection time from an aortic valve opening until a successive aortic valve closing, and two or more interbeat intervals, where the interbeat interval is the time from an aortic valve opening until a successive aortic valve opening;
(d) determining the presence of preload independence and the presence of cardiac vagal control based on the interbeat intervals;
(e) if preload independence and cardiac vagal control are present, then determining a cardiac fitness score based on the ejection time;
(f) reporting the cardiac fitness score.

10. The method of claim 9, wherein determining the presence of preload independence and the presence of cardiac vagal control comprises determining one or more measures of centrality and one or more measures of variability of two or more interbeat intervals.

11. The method of claim 10, wherein determining the presence of preload independence and the presence of cardiac vagal control comprises comparing the measures of centrality and variability to historical values for the user.

12. The method of claim 9, wherein determining the presence of preload independence comprises determining the presence of preload independence in the presence of a change in venous return to the heart.

13. The method of claim 9, wherein determining the presence of preload independence comprises comparing determining a first interbeat interval, raising a leg of the user, determining a second interbeat interval, and comparing the first and second interbeat intervals.

14. A method for determining a basal cardiac fitness of a user, comprising:
(a) providing a noninvasive sensor configured to detect changes in blood volume or flow in a measurement region of the user;
(b) providing a sensor control system configured to operate the noninvasive sensor at operational parameters to acquire a measurement signal;
(c) providing a physiological assessment system configured to determine the presence of a basal physiological state from a measurement signal;
(d) providing a trigger system configured to trigger the sensor control system to alter operational parameters if a basal physiological state is determined;
(e) providing a cardiac fitness assessment system configured to determine a cardiac fitness score from a measurement signal;
(f) using the sensor control system sensor to operate the noninvasive sensor at a first set of operational parameters to produce a first measurement signal;
(g) using the physiological assessment system to determine the presence of a basal physiological state from the first measurement signal;
(h) if a basal physiological state is determined, using the trigger system to trigger the sensor control system to alter operational parameters;
(i) using the sensor control system sensor to operate the noninvasive sensor at a second set of operational parameters to produce a second measurement signal;
(j) using the cardiac fitness assessment system configured to determine a cardiac fitness score from the second measurement signal;
(k) reporting the cardiac fitness score.

15. The method of claim 14, where the physiological assessment system is a prediction model that maps systolic time interval information contained in the first measurement signal to the presence or absence of a basal physiological state.

16. The method of claim 14, where the cardiac fitness assessment system is a prediction model that maps systolic time interval information contained in the second measurement signal to a cardiac fitness score.

17. The method of claim 15, where the prediction model comprises multiple hierarchical layers.

18. The method of claim 16, where the prediction model comprises multiple hierarchical layers.

19. A method for determining a basal cardiac fitness of a user, comprising:
(a) acquiring a first signal from a noninvasive sensor configured to detect changes in blood volume or flow in a measurement region of the user, where the measured signal contains systolic time interval information;
(b) applying a basal physiological detection model to the measured signal to determine the presence of a basal physiological state;
(c) if the presence of a basal physiological state is detected, applying a basal cardiac fitness model to the measured signal to determine a cardiac fitness score;
(d) reporting the cardiac fitness score.
